(19) United States
(12) Patent Application Publication
(10) Pub. No.: US 2002/0106688 A1
(43) Pub. Date: Aug. 8, 2002

(54) IDENTIFICATION OF GENE SEQUENCES AND GENE PRODUCTS AND THEIR SPECIFIC FUNCTION AND RELATIONSHIP TO PATHOLOGIES IN A MAMMAL

(76) Inventor: MONCEF JENBOUBI, BETHESDA, MD (US)

Correspondence Address: LYON & LYON LLP, 633 WEST FIFTH STREET, SUITE 4700, LOS ANGELES, CA, 90071, US

(21) Appl. No.: 08/906,487

(22) Filed: Aug. 5, 1997

(51) Int. Cl. G01N 33/53

(52) U.S. Cl. 435/7.1

(57) ABSTRACT

The present invention includes a basic method for discovering the function of gene and their corresponding gene products relative to a specific biological process or physiological condition. The invention provides the ability to develop therapeutic and diagnostic agents using the information obtained from the practice of the basic method. In the method, the gene product of a selected polynucleotide is delivered to a mammal to provide an immune response. The polynucleotide sequences may express, in vivo by immunization of an animal, or in bacterial system or other known system for expression of a polynucleotide sequence. The sera resulting from immunization with the gene product contains antibodies to the gene product which are used in function determinative assays to determine the function of the gene sequence gene product relative to a biological process or physiological condition, typically a disease in a human. The information derived from the function determinative assay enables the discovery of novel genes and gene products and provides the ability to design and/or manufacture of therapeutic or diagnostic products based on the practice of the basic methodology of the invention.

### FIELD OF INVENTION

The several inventions described herein are derived from a basic methodology that provides a powerful strategy to identify or discover gene sequences and gene products that are significant in a biological process or a physiological condition. Using the basic methodology provided by this invention, discrete polynucleotide sequences are identified and the gene products expressed by the polynucleotides are shown to be related to a biological process or physiological condition. This methodology provides sufficient information to define a direct, one-to-one, cause-and-effect relationship between a gene sequence, the gene product expressed by the gene sequence, and the function of that gene sequence and gene product in a specified biological process or physiological condition, typically those involved in a pathology or disease in a human. Using this information, the invention provides the ability to design and/or manufacture compounds or agents to diagnose the condition or to treat the disease. Depending on the pathology and the application of the basic methodology, the invention also provides the ability to discover or identify novel gene sequences, and novel gene products, whose specific function relative to a pathology is unknown. This invention also includes certain therapeutic agents developed or discovered based on the information obtained from the practice of the basic methodology.

### BACKGROUND

Years of research and the investment of billions of dollars have been dedicated to the attempt to discover how the DNA blueprint carried by all living things functions in both normal and abnormal states of an organism. In humans, scientists and researchers continuously struggle to understand how DNA, or its functional sub-components frequently referred to as “genes,” operates to sustain life and how the operation changes when a patient in inflicted with disease (Risch, N., and K. Merikangas. 1996. The future of genetic studies of complex human diseases. Science 273, no. 5281:1516-7.). Scientists have learned that some diseases are caused by the presence, absence or mutation of a gene (Atkinson, J., and R. Martin. 1994. Mutations to nonsense codons in human genetic disease: implications for gene therapy by nonsense suppressor tRNAs. Nucleic Acids Res 22, no. 8:1327-34; Bao, Y., P. Kishnani, J. Y. Wu, and Y. T. Chen. 1996. Hepatic and neuromuscular forms of glycogen storage disease type IV caused by mutations in the same glycogen-branching enzyme gene. J Clin Invest 97, no. 4:941-8; Bisse, E., F. Renner, S. Sussmann, J. Scholmerich, and H. Wieland. 1996. Hair iron content: possible marker to complement monitoring therapy of iron deficiency in patients with chronic inflammatory bowel diseases. Clin Chem 42, no. 8 Pt 1:1270-4; Brown, D. M., K. Vandenburgh, A. E. Kimura, T. A. Weingeist, V. C. Sheffield, and E. M. Stone. 1995. Novel frameshift mutations in the procollagen 2 gene (COL2A1) associated with Stickler syndrome (hereditary arthro-ophthalmopathy). Hum Mol Genet 4, no. 1:141-2.), while other diseases appear to coincide with changes in how certain genes operate (Bychkova, V. E., and O. B. Ptitsyn. 1995. Folding intermediates are involved in genetic diseases FEBS Lett 359, no. 1:6-8; Dean, M., and G. Santis. 1994. Heterogeneity in the severity of cystic fibrosis and the role of CFTR gene mutations. Hum Genet 93, no. 4:364-8; DeMarco, L., C. A. Stratakis, W. L. Boson, O. Jakbovitz, E. Carson, L. M. Andrade, V. F. Amaral, J. L. Rocha, G. P. Choursos, M. Nordenskjold, and E. Friedman. 1996; Sporadic cardiac myxomas and tumors from patients with Carney complex are not associated with activating mutations of the Gs alpha gene. Hum Genet 98, no. 2:185-8). Most current research attempts to correlate the existence or function of a particular gene with a specific pathological or normal physiologic condition (Giugliano, D., A. Ceriello, and G. Paolisso. 1995. Diabetes mellitus, hypertension, and cardiovascular disease: which role for oxidative stress. Metabolism 44, no. 3:363-8; Kusumi, M., K. Nakashima, H. Nakayama, and K. Takahashi. 1995. Epidemiology of inflammatory neurological and inflammatory neuromuscular diseases in Tottori Prefecture, Japan. Psychiatry Clin Neurosci 49, no. 3:169-74) and focuses on epidemiological studies of the absence or level of activity of a particular gene in certain populations or in patients suffering from a particular disease (Harper, P. S. 1995. Genetic testing, common diseases, and health service provision. Lancet 346, no. 8991-8992:1645-6; Kanekura, T., T. Kanzaki, S. Kanekura, K. Kawahara, T. Nakashima, I. Kitajima, and I. Maruyama. 1995. p53 gene mutations in skin cancers with underlying disorders. J Dermatol Sci 9, no. 3:209-14; Kondoh, M., M. Ueda, and M. Ichihashi. 1995. Correlation of the clinical manifestations and gene mutations of Japanese xeroderma pigmentosum group A patients. Br J Dermatol 133, no. 4:579-85; Lancaster, J. M., R. W. Wiseman, and A. Berchuck. 1996. An inevitable dilemma: prenatal testing for mutations in the BRCA1 breast-ovarian cancer susceptibility gene. Obstet Gynecol 87, no. 2:306-9). Some research also focuses on detailed structural studies of the gene itself, such as positional cloning, exon trapping, thorough nucleotide sequencing, and fluorescence-in-situ-hybridization (FISH) (Matsunaga, J., Y. Tomita, and H. Tagami. 1995. Detection of point mutations in human tyrosinase gene by improved allele-specific amplification. Exp Dermatol 4, no. 6:377-81; Muscatelli, F., T. M. Strom, A. P. Walker, E. Zanaria, D. Recan, A. Meindl, B. Bardoni, S. Guioli, G. Zehetner, W. Rabl, and et al. 1994. Mutations in the DAX-1 gene give rise to both X-linked adrenal hypoplasia congenita and hypogonadotropic hypogonadism. Nature 372, no. 6507:672-6). Some of these strategies have yielded limited information about certain genes and their role in disease, such as a determination that the level of activity or expression of a particular gene tends to coincide with a particular disease (Lancaster, J. M., R. W. Wiseman, and A. Berchuck. 1996. An inevitable dilemma: prenatal testing for mutations in the BRCA1 breast-ovarian cancer susceptibility gene. Obstet Gynecol 87, no. 2:306-9.), or that a gene implicated in a disease is located on a particular chromosome (Muscatelli, F., T. M. Strom, A. P. Walker, E. Zanaria, D. Recan, A. Meindl, B. Bardoni, S. Guioli, G. Zehetner, W. Rabl, and et al. 1994. Mutations in the DAX-1 gene give rise to both X-linked adrenal hypoplasia congenita and hypogonadotropic hypogonadism. Nature 372, no. 6507:672-6.). In other cases, exhaustive efforts by teams of scientists and researchers have led to the putative identification of a gene that may be a cause of, or which may predispose a person to, a particular disease (Parry, D. M., M. M. MacCollin, M. I. Kaiser-Kupfer, K. Pulaski, H. S. Nicholson, M. Bolesta, R. Eldridge, and J. F. Gusella. 1996. Germ-line mutations in the neurofibromatosis 2 gene: correlations with disease severity and retinal abnormalities. Am J Hum Genet 59, no. 3:529-39; Ritvaniemi, P., J. Korkko, J. Bonaventure, M. Vikkula, J. Hyland, P. Paassilta, I. Kaitila, H. Kaariainen, B. P. Sokolov, M. Hakala, and et al. 1995. Identification of COL2A1 gene mutations in patients with chondrodysplasias and familial osteoarthritis. Arthritis Rheum 38, no. 7:999-1004; Pospelov, L. E., A. G. Matrakshin, L. N. Chernousova, K. N. Tsoi, K. I. Afanasjev, G. A. Rubtsova, and V. V. Yeremeyev. 1996. Association of various genetic markers with tuberculosis and other lung diseases in Tuvinian children. Tuber Lung Dis 77, no. 1:77-80).

However, the mere identification of a particular gene sequence as being related to a given disease, based for example on the identification of the nucleotide sequence of the gene or a mutation thereof as being present in patients with a given pathology, does not provide any information about how the expression of the gene sequence is related to the progression of the disease, nor is it usually possible to determine how, when, or where the product expressed by the gene sequence functions in the specific pathology (Orr, H. T., and H. B. Clark. 1995. Genetic approaches to pathogenesis of neurodegenerative diseases. Lab Invest 73, no. 2:161-71; Smith, G. K., J. Jie, G. E. Fox, and X. Gao. 1995. DNA CTG triplet repeats involved in dynamic mutations of neurologically related gene sequences form stable duplexes. Nucleic Acids Res 23, no. 21:4303-11). Moreover, such information does not reveal whether the gene product causes or contributes to a disease, does not reveal whether or not a particular gene product is localized or is present in varying amounts during the initiation or progression of a disease, and, most importantly, does not identify the gene sequences or gene products that are principal causes of a disease.

For example, using traditional research methodology, scientists may invest thousands of hours and millions of dollars to determine that one particular gene is highly expressed in patients who have a particular physiological condition, or to the contrary, that the gene is essentially non-functional in patients suffering from a particular disease (Vesa, J., E. Hellsten, L. A. Verkruyse, L. A. Camp, J. Rapola, P. Santavuori, S. L. Hofmann, and L. Peltonen. 1995. Mutations in the palmitoyl protein thioesterase gene causing infantile neuronal ceroid lipofuscinosis. Nature 376, no. 6541:584-7). In such a case, scientists or researchers have merely determined that one identifiable affect on a particular gene is coincident to a certain disease. However, even where such information is available, and the available of such information is generally regarded as a scientific triumph, the practical utility of such information may be extremely limited because the discovery of such information does not equate to a discovery that the expression of the gene is the actual cause of the disease, nor can the conclusion be made that disruption of the expression of a highly expressed gene, or disruption of the function of the expressed gene product, will prevent or alleviate the disease. Similarly, the conclusion cannot be drawn that restoring the expression of a gene that is not expressed in a particular gene state, or providing a substitute for the normally expressed gene product, will convey any clinical benefit (Alton, E. W., and D. M. Geddes. 1995. Gene therapy for cystic fibrosis: a clinical perspective. Gene Ther 2, no. 2:88-95; Blaese, R. M., K. W. Culver, A. D. Miller, C. S. Carter, T. Fleisher, M. Clerici, G. Shearer, L. Chang, Y. Chiang, P. Tolstoshev, and et al. 1995. T lymphocyte-directed gene therapy for ADA-SCID: initial trial results after 4 years. Science 270, no. 5235:475-80; Correll, P. H., and S. Karlsson. 1994. Towards therapy of Gaucher's disease by gene transfer into hematopoietic cells. Eur J Haematol 53, no. 5:253-64).

In some cases, scientists have identified human genes that are implicated in a disease or pathologic condition and have located similar genes in mice or other animal species (Mansour, S. L., Thomas, K. R., and Capecchi, M. R. Disruption of the proto-oncogene int-2 in mouse embryo-derived stem cells: a general strategy for targeting mutations to non-selectable genes. Nature 336: 348-352, 1988; Aiba, A., M. Kano, C. Chen, M. E. Stanton, G. D. Fox, K. Herrup, T. A. Zwingman, and S. Tonegawa. 1994. Deficient cerebellar long-term depression and impaired motor learning in mGluR1 mutant mice. Cell 79, no. 2:377-88). Attempts to verify the structure and function of the identified animal gene to determine information that is truly useful for human therapy, using animal genetics, has resulted in contradictory and unpredictable results. Often, these findings are of limited utility in human medicine and can even complicate the understanding of the physiological role of a newly discovered gene in humans (Mansour, S. L., Thomas, K. R., and Capecchi, M. R. Disruption of the proto-oncogene int-2 in mouse embryo-derived stem cells: a general strategy for targeting mutations to non-selectable genes. Nature 336: 348-352, 1988; Aiba, A., M. Kano, C. Chen, M. E. Stanton, G. D. Fox, K. Herrup, T. A. Zwingman, and S. Tonegawa. 1994. Deficient cerebellar long-term depression and impaired motor learning in mGluR1 mutant mice. Cell 79, no. 2:377-88).

Thus, an important barrier to the treatment of disease is the current state of the art to identify a specific gene sequence or gene product that plays a dispositive role in causing a disease. Furthermore, in the vast majority of cases, scientists presently cannot identify a gene sequence or gene product capable of preventing or alleviating a given disease (Mansour, S. L., Thomas, K. R., and Capecchi, M. R. Disruption of the proto-oncogene int-2 in mouse embryo-derived stem cells: a general strategy for targeting mutations to non-selectable genes. Nature 336: 348-352, 1988; Aiba, A., M. Kano, C. Chen, M. E. Stanton, G. D. Fox, K. Herrup, T. A. Zwingman, and S. Tonegawa. 1994. Deficient cerebellar long-term depression and impaired motor learning in mGluR1 mutant mice. Cell 79, no. 2:377-88). Thus, traditional research methodology predominantly does not provide the capability to define a direct, one-to-one relationship between a gene sequence or gene product and a particular biological process or physiological condition such that a cause and effect relationship can be established between the process or condition and the expression of the gene sequence.

Furthermore, any attempt to establish a direct, one-to-one cause-and-effect relationship between a gene sequence and a disease using traditional methodology is further frustrated by the discovery that more than one gene, and as many as hundreds of genes, may be expressed to either a greater or lesser degree in patients with a particular disease. Except in unusual circumstances, the scientist or researcher cannot know whether the over- or under-expression of a gene sequence, and the resulting presence or absence of the corresponding gene product, is actually causing the disease, is part of the body's attempt to counteract the disease, or is part of a more complex cascade of biological processes that merely accompany the disease.

Furthermore, the product of a particular gene sequence may perform different functions in different tissue types, may perform different functions at different stages of a given disease, may perform different functions depending on the particular form of the gene product that is expressed, and may perform different functions depending on whether the gene product is secreted from a cell or functions in the intracellular space. Thus, the identification of a gene sequence or gene product as being merely coincident to a disease or physiological condition does not provide adequate information to intervene clinically to cure or alleviate the specific disease or condition of interest.

Even where a gene is identified and the function is known, the ability to develop a therapeutic strategy from this information is frustrated because of the lack of information on the precise relationship between the gene sequence, the gene product, and the actual causation of the pathology. Therefore, even where very specific information correlating the function of a gene sequence to a particular disease is known, determining the relationship between the gene sequence, the gene product, the specific pattern of expression of the gene, the specific location of the gene product in the affected tissues, and determining how each of the above affects the clinical outcome of a disease may not be possible. Without this knowledge, a meaningful understanding of the specific relationship between the gene itself, the gene product, and a pathological condition is not achieved, and the ability to develop a treatment or cure for the condition does not exist or is fatally impaired. Virtually no strategy exists that allows a researcher to readily discover or identify specific gene sequences having a one-to-one relationship with gene products that exhibit cause-and-effect relationship with a selected biological process or physiological phenomenon.

The above situation also plagues the effort to discover the function of genes or gene sequences that can be isolated by chemical methods, but about which virtually nothing is known regarding their specific function or role in normal or abnormal biochemical processes or physiological conditions. For example, the prospect of determining the DNA sequence of the entire mammalian genome (usually known as the Human Genome Project) has led to a great hope for discovering new genes that cause or predispose a patient to a variety of diseases. However, simply knowing the nucleotide sequence of the entire human genome does not directly lead to therapeutic applications because of the absence of an adequate methodology to identify gene sequences having a specific function relative to a selected biochemical process or physiological condition.

Therefore, the current strategies that attempt to correlate a gene sequence to a specific pathology are unable to provide a meaningful understanding of the specific function of a given gene sequence and its specific pattern of expression relative to disease. In addition, many of these strategies produce only limited information and are prohibitively labor intensive (Lancaster, J. M., R. W. Wiseman, and A. Berchuck. 1996. An inevitable dilemma: prenatal testing for mutations in the BRCA1 breast-ovarian cancer susceptibility gene. Obstet Gynecol 87, no. 2:306-9; Muscatelli, F., T. M. Strom, A. P. Walker, E. Zanaria, D. Recan, A. Meindl, B. Bardoni, S. Guioli, G. Zehetner, W. Rabl, and et al. 1994. Mutations in the DAX-1 gene give rise to both X-linked adrenal hypoplasia congenita and hypogonadotropic hypogonadism. Nature 372, no. 6507:672-6; Ritvaniemi, P., J. Korkko, J. Bonaventure, M. Vikkula, J. Hyland, P. Paassilta, I. Kaitila, H. Kaariainen, B. P. Sokolov, M. Hakala, and et al. 1995. Identification of COL2A1 gene mutations in patients with chondrodysplasias and familial osteoarthritis. Arthritis Rheum 38, no. 7:999-1004). Therefore, a need exists for the capability to determine how the function of a gene sequence or gene product determines the outcome of a biochemical process or physiological condition, including establishing a cause and effect relationship between the expression of the gene sequence and the existence or progression of a disease. A need also exists for a technique to discover new genes and gene products based on, and starting with, the ability to obtain information about how the expression of a gene product affects the existence or progression of a biological process or physiological condition. A need also exists for the ability to analyze thoroughly the gene sequences that comprise the human genome, to identify their gene products, and to determine how the gene products function in different cell types, such as cancer cell lines, primary cell lines, and tissue specific cell lines.

There is also a great need discover previously unrecognized genes that may control a variety of well-known and threatening disorders that are currently known to have a genetic component such as cardiovascular disease, cancer, inflammatory conditions, degenerative diseases, neurological and infectious diseases, among numerous other pathologies. A need also exists to discover expression patterns for gene sequences that have already been identified but whose function is poorly understood and that will significantly control the initiation or progression of disease. Therefore, a need exists to generate and assemble information related to mammalian genes, their pattern of expression, their gene products and their derivatives, and their cellular expression at both the tissular and temporal level. Such a databank would greatly benefit scientific research in academia, private research centers, and in commercial enterprise.

Accordingly, an invention which provided the ability to discover or identify a gene sequence which, by virtue of the expression of the corresponding gene product, has a direct, one-to-one, cause-and-effect relationship with a biological process or physiological condition would be extremely valuable to researchers who could use the information to discover important new gene sequences and gene products, to increase the sum of knowledge regarding the function of genes and their relationship to disease, and to develop therapeutic products to diagnose or to cure disease based on this information. Specifically, when a one-to-one relationship is established between a gene sequence, the product of that gene sequence, and the cause and effect relationship between the gene sequence or gene product and a biological process of physiological condition, researchers can use the knowledge of that relationship to diagnose or treat the process or condition in a patient, principally by developing treatment strategies and therapeutic agents to affect the function or operation of the gene product. For example, using the information obtained from this invention, a beneficial gene product can readily be manufactured in large quantities by known techniques; similarly, compounds or agents that mimic certain aspects of the gene product or derivatives thereof, can be produced. Antibodies can also be manufactured to alter the function of a gene product by binding to the gene product directly or by binding to a compound or structure, such as a cell surface marker, with which a gene product may interact. Many such strategies are described or suggested below and additional approaches will be readily apparent to those of skill in the art.

### DESCRIPTION OF THE INVENTION

The present invention uses a partial or complete naked polynucleotide (DNA or RNA), or a combination of partial or complete naked polynucleotide sequences to obtain controlled expression of the gene product, or any of its derivatives, encoded by the polynucleotide. In one embodiment, expression is achieved by placing the polynucleotide(s) under the control of eukaryotic regulatory elements in a plasmid construct administered to a mammal (For illustrative methodology relating to obtaining antibody-encoding polynucleotides, see Ward et al., Nature, 341:544-546 (1989); Gillies et al., Biotechnol. 7:799-804 (1989); and Nakatani et al., loc. cit., 805-810 (1989). In an alternate embodiment, the gene product, and/or its derivatives, of the selected polynucleotide sequence or combination of polynucleotide sequences may be obtained by other mechanisms, such as by the use of a prokaryotic expression system as described herein. The gene product(s) expressed by the polynucleotide(s) are administered to the functional components of a mammalian immune system to perform an immunization that yields an immune response that generates immunoglobulins that are specific to the gene product and/or its derivatives.

Where the polynucleotide is present in a plasmid administered to the mammal, the method of immunization with the gene product is achieved in vivo by virtue of the expression of the gene product from the polynucleotide contained in the plasmid. Where the gene product is expressed outside the mammal to be immunized, the gene product(s) or derivative thereof, is administered to the mammal to achieve immunization. Moreover, although the immunization step described herein is preferably achieved by administration of the gene product to the intact immune system of a living mammal, any other in vivo or in vitro technique that exposes an ontogenic gene product to the functional components of a mammalian immune system may be used to obtain immunoglobulins pursuant to this invention.

These immunoglobulins are comprised of monospecific polyclonal antibodies, sometimes referred to herein as gene product tags (GPTs), are specific to the gene product(s) and/or its derivatives encoded by the selected polynucleotide(s). Where an immunized mammal is used, the mammal's sera containing the immunoglobulins is then used in function determinative assays comprised of at least one immunoassay, to reveal information about a selected biological process or physiological condition. By relating the outcome of the immunoassay and other testing that may comprise the function determinative assays, to the specific gene sequence from which the corresponding antibody to the product expressed by the gene sequence generated the results of interest in the assay(s), a discrete polynucleotide or gene sequence is identified and a direct, one-to-one, relationship is established between the function determinative result obtained in the assay, the polynucleotide, or gene sequence, the gene product expressed therefrom, and the corresponding antibody (Justewicz, D. M., and R. G. Webster. 1996. Long-term maintenance of B cell immunity to influenza virus hemagglutinin in mice following DNA-based immunization. Virology 224, no. 1:10-7; Krasemann, S., M. Groschup, G. Hunsmann, and W. Bodemer. 1996. Induction of antibodies against human prion proteins (PrP) by DNA-mediated immunization of PrPO/O mice. J Immunol Methods 199, no. 2:109-18).

When the mammal's immune system is exposed to a plurality of gene products, this methodology provides a high capacity, high efficiency, screening process that identifies polynucleotides corresponding to a complete or partial sequence of a coding gene for which a direct, one-to-one relationship exists between the polynucleotide, the corresponding gene product and/or its derivative, and the biological process or physiological condition tested by the immunoassay (Lopez-Macias, C., M. A. Lopez-Hernandez, C. R. Gonzalez, A. Isibasi, and V. Ortiz-Navarrete. 1995. Induction of antibodies against Salmonella typhi OmpC porin by naked DNA immunization. Ann N Y Acad Sci 772:285-8; Luke, C. J., K. Carner, X. Liang, and A. G. Barbour. 1997. An OspA-based DNA vaccine protects mice against infection with Borrelia burgdorferi. J Infect Dis 175, no. 1:91-7). Accordingly, this invention provides the ability to screen polynucleotides to identify discrete gene sequences and corresponding gene products that are related to any biological process or physiological condition that may be tested by an immunoassay will be highly useful, for example, to identify and determine gene products implicated in disease (Lu, S., J. Arthos, D. C. Montefiori, Y. Yasutomi, K. Manson, F. Mustafa, E. Johnson, J. C. Santoro, J. Wissink, J. I. Mullins, J. R. Haynes, N. L. Letvin, M. Wyand, and H. L. Robinson. 1996. Simian immunodeficiency virus DNA vaccine trial in macaques. J Virol 70, no. 6:3978-91; Major, M. E., L. Vitvitski, M. A. Mink, M. Schleef, R. G. Whalen, C. Trepo, and G. Inchauspe. 1995. DNA-based immunization with chimeric vectors for the induction of immune responses against the hepatitis C virus nucleocapsid. J Virol 69, no. 9:5798-805), to identify gene sequences whose up- or down-regulation has a direct relationship to a disease, to determine whether a gene product is present or absent in certain cell lines in normal or pathological conditions, and to identity and determine gene sequences and gene products whose function is outcome determinative of a disease. Using this screening aspect of the methodology, novel gene sequences and novel gene products can also be identified and the specific function and mechanism of novel gene sequences and gene products can be determined based on the action of the antibodies (GPTs) to the gene products in function determinative assays related to any biological process or physiological condition.

The methodology of the invention is also useful where the gene or gene product has previously been attributed to an important biological or physiological function, or to a specific disease, because additional information regarding isoforms, mutations, or patterns of expression may be obtained. Also, even when the expression or existence of the gene product does not control a disease, the specific polynucleotide or gene product identified or obtained by the practice of the basic methodology of the invention, may be useful to diagnose a disease, such as where a specific gene product is present in a disease state, for example, the gene product may be a cell surface marker on a cancer cell. Essentially, the invention is useful where the polypeptide expression product of a polynucleotide coincides with, identifies, prevents, controls, contributes to, retards, or cures a disease or physiological condition having a cause or effect that may be determined by identifying a basis in the selective expression of a polynucleotide, such as a cancer (occurring in any type of tissue), as well as other conditions involving inflammation, cardiovascular disease, neurodegenerative disease, psychiatric disorders, aging, immunological disfunction, and infectious diseases (Miki, Y., J., D. Swensen, and e.a. Shattuck-Eidens. 1994. A strong candidate for the breast and ovarian cancer susceptibility gene BRCA1. Science 266:66-71; Zhang. Y, Proenca. R, M. M., B. M., Leopold. L, and F. J. M. 1994. Positional cloning of the mouse obese gene and its human homologue. Nature 372:425-428; Seth, A., D. Palli, J. M. Mariano, R. Metcalf, M. C. Venanzoni, S. Bianchi, S. D. Kottaridis, and T. S. Papas. 1994. P53 gene mutations in women with breast cancer and a previous history of benign breast disease. Eur J Cancer 30A, no. 6:808-12; Strijbos, P. J., and N. J. Rothwell. 1995. Interleukin-1 beta attenuates excitatory amino acid-induced neurodegeneration in vitro: involvement of nerve growth factor. J Neurosci 15, no. 5 Pt 1:3468-74; Piscitelli, S. C., and J. R. Minor. 1995. Role of interleukin-2 in managing infection with the human immunodeficiency virus. Am J Health Syst Pharm 52, no. 5:541-2).

As note above, the basic methodology of this invention is preferably comprised of delivering to the immune system of a mammal, preferably a non-human mammal, usually a rodent and most preferably a mouse, the expression product or “gene product” of one or more complete or partial polynucleotide (DNA or RNA) sequences. The gene product may be obtained from the polynucleotide by placing the polynucleotide under the control of a suitable promoter and enhancer, i.e., in a plasmid construct and administering the construct to the functional components of a mammalian immune system (Starling, G. C., M. B. Llewellyn, G. S. Whitney, and A. Aruffo. 1997. The Ly-1.1 and Ly-1.2 epitopes of murine CD5 map to the membrane distal scavenger receptor cysteine-rich domain. Tissue Antigens 49, no. 1:1-6; Pande, H., K. Campo, B. Tanamachi, S. J. Forman, and J. A. Zaia. 1995. Direct DNA immunization of mice with plasmid DNA encoding the tegument protein pp65 (ppUL83) of human cytomegalovirus induces high levels of circulating antibody to the encoded protein. Scand J Infect Dis Suppl 99:117-20; Ragno, S., M. J. Colston, D. B. Lowrie, V. R. Winrow, D. R. Blake, and R. Tascon. 1997. Protection of rats from adjuvant arthritis by immunization with naked DNA encoding for mycobacterial heat shock protein 65, Arthritis Rheum 40, no. 2:277-83). The polynucleotide may code for a known, an unknown, or a combination of known and unknown gene products. The polynucleotide may be originally prepared from an isolated cDNA library and is usually derived from a specific mammalian tissue (Maniatis, T., Fritsch, E. F. and Sambrook, T. (1982). Molecular Cloning: A Laboratory Manual. Cold Spring Harbor Lab, Cold Spring Harbor, N.Y.), and is preferably derived from human DNA. In many applications of the invention, the polynucleotide is derived from human DNA obtained from cells or tissues reflecting a specific pathological condition.

The polynucleotide can be generated using any of several techniques that are well known in the art of molecular biology, such as alkaline lysis, cesium chloride gradient and quiagen DNA preparation (Maniatis, T., Fritsch, E. F. and Sambrook, T. (1982). Molecular Cloning: A Laboratory Manual. Cold Spring Harbor Lab, Cold Spring Harbor, N.Y.). Also, as noted above, the gene product may be obtained by any known technique that expresses the polynucleotide to obtain the gene product (Maniatis, T., Fritsch, E. F. and Sambrook, T. (1982). Molecular Cloning: A Laboratory Manual. Cold Spring Harbor Lab, Cold Spring Harbor, N.Y.). For example, known bacterial expression systems may be used to obtain the gene product of the polynucleotide sequence.

The polynucleotide is preferably in its naked form, and may be a partial or complete polynucleotide sequence from a known or unknown gene. The process of immunization may use one polynucleotide or a pool of more than 1, such as 2, 3, 4, 5, 6, 7, 8, 9, or 10 clones or more, preferably between 5 and 10, where the ranges may vary to integral values the between according to the needs of the invention. Also, prior to the immunization, a variety of known techniques can be employed to limit the total number of polynucleotides and corresponding gene products used in the immunization step to reduce the redundancy of gene sequences expressed at high levels and to increase the capacity and efficiency of the methodology. In many cases, the source of the polynucleotides is a specific cell line such as T-cells, hematopoietic cells, tumor cells, etc. from which known redundant gene sequences can be removed by a variety of techniques (Maniatis, T., Fritsch, E. F. and Sambrook, T. (1982). Molecular Cloning: A Laboratory Manual. Cold Spring Harbor Lab, Cold Spring Harbor, N.Y.). Subtractive hybridization may also be employed to remove polynucleotide sequences that are known to be extraneous, such as “housekeeping” genes (Maniatis, T., Fritsch, E. F. and Sambrook, T. (1982). Molecular Cloning: A Laboratory Manual. Cold Spring Harbor Lab, Cold Spring Harbor, N.Y.). Where a biological process or physiological condition is analyzed that involves an abnormal or mutant cell or tissue representing a selected pathological condition, DNA from the normal, wild-type cells and/or tissue may be subtracted from the abnormal cells and/or tissue to enrich the polynucleotide pool in sequences specific for the pathological condition.

Immunization of a mammal with a plasmid construct is preferably achieved by injection, i.e., intravenous, intramuscular, or intraperitoneal, and may also be achieved by inhalation, suppository, or dermal/occular application (Bourne, N., G. N. Milligan, M. R. Schleiss, D. I. Bernstein, and L. R. Stanberry. 1996. DNA immunization confers protective immunity on mice challenged intravaginally with herpes simplex virus type 2. Vaccine, 14 no. 13:1230-41; Lawson, C. M., J. R. Bennink, N. P. Restifo, J. W. Yewdell, and B. R. Murphy. 1994. Primary pulmonary cytotoxic T lymphocytes induced by immunization with a vaccinia virus recombinant expressing influenza A virus nucleoprotein peptide do not protect mice against challenge. J Virol 68, no. 6:3505-11; Vadolas, J., J. K. Davies, P. J. Wright, and R. A. Strugnell. 1995. Intranasal immunization with liposomes induces strong mucosal immune responses in mice. Eur J Immunol 25, no. 4:969-75; Winegar, R. A., J. A. Monforte, K. D. Suing, K. G. O'Loughlin, C. J. Rudd, and J. T. Macgregor. 1996. Determination of tissue distribution of an intramuscular plasmid vaccine using PCR and in situ DNA hybridization. Hum Gene Ther 7 no. 17:2185-94; Yokoyama, M., J. Zhang, and J. L. Whitton. 1996. DNA immunization: effects of vehicle and route of administration on the induction of protective antiviral immunity. FEMS Immunol Med Microbiol 14, no. 4:221-30). The plasmid constructs may include elements to facilitate integration into the genome of the mammal that is immunized, for example, the plasmid construct may include, or be associated with, transfection facilitating viral particles, liposomal formulations, or charged lipids, and may include infection or integrating DNA sequences. The plasmid constructs may also include elements to allow the secretion of the gene product in the intercellular space and to facilitate its uptake by antigen presenting cells (APCs) (Germain, R. N. and Margulies, D. H. The biochemistry and cell biology of antigen processing and presentation. 1993. Annu Rev Immunol 11: 403-450; Cresswell, P. and Blum, J. S. Intracellular transport of class II HLA antigens. 1988. In B. Pernis, S. C. Silverstein, and H. J. Vogel (eds.): Processing and Presentation of Antigens, pp. 43-51, Calif. Academic, San Diego, Calif.). The plasmid constructs may also include elements that will increase the immunogenicity of the gene product.

The mammalian immune system possesses a large capacity to produce an extremely diverse and distinct number of highly specific immunoglobulins (Tonegawa, S. 1993. The Nobel Lectures in Immunology. The Nobel Prize for Physiology or Medicine, 1987. Somatic generation of immune diversity [classical article]. Scand J Immunol 38, no. 4:303-19; Winter. G, and M. C. 1991. Man-made antibodies. Nature 349:293-299), estimated at 10¹² different immunoglobulin molecules. The different strategies for the production of antibodies, the availability of simple and reliable assays to detect their specificity, and their effect on mammalian cells, offers a unique advantage for their use as a tool to characterize a particular gene product administered to a mammalian immune system (Janeway, C. A., Jr., Conrad, P. J., Lerner, E. A., Babich, J., Wettstein, P., and Murphy, D. B. 1984. Monoclonal antibodies specific for Ia glycoproteins raised by immunization with activated T cells: possible role of T cellbound Ia antigens as targets of immunoregulatory T cells. J Immunol 132: 662-667; Ozato, K. and Sachs, D. H. 1981. Monoclonal antibodies to mouse MHC antigens. III. Hybridoma antibodies reacting to antigens of the H-2b haplotype reveal genetic control of isotype expression. J Immunol 126: 317-321). By generating sera containing immunoglobulins against the gene product of a known or an unknown gene sequence, the specific binding of the immunoglobulin in a function determinative assay identifies the localization and function of the gene product, and hence the identity and function of the gene sequence itself, by virtue of the interaction between the gene product and the specific antibody. Therefore, the specificity of the anti-gene product immunoglobulin and its reaction with the gene product identifies the precise localization and function of the gene product and can demonstrate the gene product. The binding reaction of the gene product and the immunoglobulin molecule, whether intra or extracellular, can also demonstrate the ability to identity, initiate, modulate, or activate or block either structurally or functionally, a cell component, such as a cell surface receptor, or any biologically active compound, such as a hormone or growth factor (Bessis, N., D. Fradelizi, C. Fournier, and M. C. Boissier. 1995. [Treatment of inflammation with anti-inflammatory cytokines: example in models of auto-immune diseases]. C R Seances Soc Biol Fil 189, no. 4:579-90; Fukase, S., N. Ohta, K. Inamura, Y. Kimura, M. Aoyagi, and Y. Koike. 1994. Diagnostic specificity of anti-neutrophil cytoplasmic antibodies (ANCA) in otorhinolaryngological diseases. Acta Otolaryngol Suppl (Stockh) 511:204-7; Karlsson-Parra, A., T. Skogh, and Z. Heigl. 1994. [Antinuclear antibodies. Diagnostic potential in systemic rheumatic diseases]. Lakartidningen 91, no. 4:245-9; Kashima, K., S. Oka, A. Tabata, K. Yasuda, A. Kitano, K. Kobayashi, and I. Yano. 1995. Detection of anti-cord factor antibodies in intestinal tuberculosis for its differential diagnosis from Crohn's disease and ulcerative colitis. Dig Dis Sci 40, no. 12:2630-4).

As part of the screening process, and to reduce the overall number of immunizations while maintaining the ability to isolate the particular polynucleotides that yield results in an immunoassay, individual clones may be located in a plurality of wells oriented in a grid or in an array as in a conventional multi-well plate. For example, given a conventional multi-well plate with a single clone dedicated to each well, a single immunization is made either with a single clone or a pool of clones comprised of individual clones contained in each well along each horizontal row of wells, followed by an immunization comprised of a pool of clones from each well along each vertical axis. As noted above, the sera obtained from each immunization are used in a function determinative assay comprised of at least one immunoassay, and optimally containing additional immunoassays or other non-immunoassays that yield information about a biological process or physiological phenomenon (Bessis, N., D. Fradelizi, C. Fournier, and M. C. Boissier. 1995. [Treatment of inflammation with anti-inflammatory cytokines: example in models of auto-immune diseases]. C R Seances Soc Biol Fil 189, no. 4:579-90; Fukase, S., N. Ohta, K. Inamura, Y. Kimura, M. Aoyagi, and Y. Koike. 1994. Diagnostic specificity of anti-neutrophil cytoplasmic antibodies (ANCA) in otorhinolaryngological diseases. Acta Otolaryngol Suppl (Stockh) 511:204-7; Karlsson-Parra, A., T. Skogh, and Z. Heigl. 1994. [Antinuclear antibodies. Diagnostic potential in systemic rheumatic diseases]. Lakartidningen 91, no. 4:245-9; Kashima, K., S. Oka, A. Tabata, K. Yasuda, A. Kitano, K. Kobayashi, and I. Yano. 1995. Detection of anti-cord factor antibodies in intestinal tuberculosis for its differential diagnosis from Crohn's disease and ulcerative colitis. Dig Dis Sci 40, no. 12:2630-4).

Typically, selected immunoassay(s) will be repeated many times until the desired results are achieved with an antibody that corresponds to a single polynucleotide. At that point, a one-to-one correlation is established between a gene sequence, consisting of the cDNA clone, the gene product, the antibody to the gene product, and the function determined by the assay. When the desired results are seen in one or more of the assays, for example, when a pool of clones are used, the pool of clones which yielded the desired result can be cross-referenced by repeating the immunization using a subdivision of the original clones. Similarly, the assay can be repeated using each clone from a given pool or subcombinations thereof to identify one, or a small number of polynucleotide sequences demonstrating the desired results in the function determinative assays.

Therefore, the results of the immunoassay demonstrate that an antibody to the gene product was able to affect the biological process of physiological function tested by the assay. For example, an immunoassay may demonstrate the ability of the sera obtained from the immunized mammal to substantially reduce the growth of tumor cells. The results of such an immunoassay would thereby indicate that the binding of an antibody specific to the gene product expressed by a polynucleotide reduces the growth of tumor cells in the assay. Thus, the information obtained from the practice of the basic methodology of the invention may identify a gene product that is necessary for the growth of tumor cells or may function by another mechanism. In any case, examination of the gene sequence identified by the basic methodology of the invention reveals the identity and nature of the gene product. Thus, the practice of the basic methodology of the invention, and the information derived therefrom, establish a one-to-one correlation between the gene sequence, the gene product, and the function of the gene product in the cell type and the pathological condition tested by the assays. Together, this information provides the ability to obtain a therapeutic product or strategy that can be readily obtained by conventional methods.

The “function determinative” assays as described herein includes at least one immunoassay, and numerous assays described in the examples, see, e.,g., Examples 17 through 37. Thus, depending on the nature of the biological process or physiological condition being examined, the immunoassay may be accompanied by one or more additional assays selected to yield significant information relative to the process or condition tested, see e.g., Examples 38 through 56 below. Thus, the scientist selects those assays where the certain results of the assay logically provides information to determine the function of a gene product through the action of the corresponding antibody. In this manner, the basic methodology of the invention identifies a polynucleotide and corresponding gene product having a direct, one-to-one relationship to the biological process or physiological condition of interest. This one-to-one relationship links directly the gene sequence, the gene product, the antibody to the gene product (GPT), the cellular localization, the cell type, the molecular weight, and the biological process or physiological condition, which, in turn, is applicable to a disease or pathology of interest. The immunoassays and function determinative assays that are available for use in this invention are numerous and well known in the art. Also, the ability to select the particular assays to be utilized in the basic methodology of the invention to yield the desired information about a gene product relative to a disease or pathology is well within the capability of those of ordinary skill in the art. Most frequently, the functional assays relied upon in the context of any particular analysis, for example, a tumor in a selected cell line, will be the assays that are currently used most often in traditional research on the same subject matter. The advantage provided to the present invention is the ability to identify the polynucleotide sequence and corresponding gene product that have a direct, one-to-one relationship to the process or condition tested.

As noted above, once the results are obtained from the assay(s), a single clone is identified and the actual polynucleotide sequence is examined in detail to determine the nature of the sequence and gene product encoded thereby. The basic methodology of the invention may be practiced to this point only, and highly valuable information is still obtained. In the above example, a polynucleotide may be discovered that codes for a surface marker present on particular tumor cells, antibodies specific to the gene product of that polynucleotide will bind to the cell surface marker. If the binding of the antibody to the cell surface marker of the tumor cell retards growth of the tumor, the antibody has direct therapeutic interest. Even if the antibody does not directly affect tumor growth, identifying a specific polynucleotide that encodes a cell surface marker unique to a type of tumor cell is potentially valuable as a diagnostic product, enhances understanding of the progression of the disease, and may provide means for therapeutic intervention involving compounds that are combined with the antibody such as radioactive or chemotherapeutic agents. Furthermore, additional assays or tests may be performed to obtain additional information regarding the gene product or gene sequence. The assays may reveal information about ion exchange across the cell membrane, the pathways of cell growth or metastasis, or other biochemical process or pathway involved in the cell function. This additional information complements the information obtained from at least one immunoassay of the function determinative assay(s) and adds to the understanding of the biological process or physiological condition of interest. Therefore, the totality of the methodology of the present invention may be comprised of numerous steps in addition to the basic methodology, however, valuable information may be obtained by performing far less than all of the conceivable steps described herein.

In many cases, in addition to the basic immunoassay, additional assays or analysis of different polynucleotide samples may provide more detailed and valuable information that applies specifically to the development of a clinical therapeutic product. As described in more detail below, any gene sequence, fragment of a gene sequence, gene product, antibodies to a gene product or any of their derivatives or compounds that affect the function of any of the above may be developed as therapeutic agents based on the information obtained from the practice of the basic methodology of the invention. Moreover, the advantages gained by this invention may be exploited by beginning with only the information obtained by the practice of the basic methodology. Thus, once a gene sequence and gene product is identified by the basic methodology, the fruits and benefits of the invention may be enjoyed by developing a therapeutic product by conventional methods based on information which would not exist if not for the practice of the basic methodology. Moreover, as additional such assays are developed by scientists, any newly developed assay can be utilized in the basic methodology of this invention. The use of newly developed assays will increase the utility of the invention and enhance the discovery of gene products and the possibility of therapeutic intervention.

In the above example, the results of the immunoassay indicate that an antibody in the sera of the immunized mammal has reacted in a manner such that the result of the assay provides information about biological process or physiological function being tested by the immunoassay. The antibody binding reaction can occur in any number of ways depending on the type of assay, the process or condition being tested by the assay, the nature of the gene product, and the nature of the antibody contained in the sera. For example, the antibody may bind to a cell surface marker, thus causing the cell to grow, die, differentiate, or perform any other cellular function controlled by such binding. The antibody may block the binding of a substance that performs the above functions or others. The antibodies may also cause the removal of compounds that cause such actions or may enhance their function. Furthermore, the antibodies may block cell-to-cell interactions, may cause or prevent apoptosis, cellular or tissular degeneration, cellular or tissular regeneration, cell differentiation, or any other cellular function (Bush, R. K., and H. Sanchez. 1997. In vitro synthesis of Alternaria allergens and their recognition by murine monoclonal and human IgE antibodies. Ann Allergy Asthma Immunol 78, no. 3:287-92; Fuller, D. H., and J. R. Haynes. 1994. A qualitative progression in HIV type 1 glycoprotein 120-specific cytotoxic cellular and humoral immune responses in mice, receiving a DNA-based glycoprotein 120 vaccine. AIDS Res Hum Retroviruses 10, no. 11:1433-41; Gonzalez Armas, J. C., C. S. Morello, L. D. Cranmer, and D. H. Spector. 1996. DNA immunization confers protection against murine cytomegalovirus infection. J Virol 70, no. 11:7921-8).

Another highly useful aspect of the invention derives from the ability to determine the localization at which the binding of the antibody has caused the outcome of the assay. This is achieved by either labelling the antibodies used in the assays where a useful result is achieved or by using a labelled anti-antibody to visualize the antibody to the gene product. This procedure may be accomplished in several ways that are well known to those of ordinary skill in the art. For example, if the immunized mammal is a mouse, and thus murine sera was used in the assays, labelled anti-mouse antibodies, i.e., anti-mouse antibodies coupled with horseradish peroxidase, alkaline phosphatase, or fluorochacomes, are used to bind specifically to the mouse antibody contained in the sera. These labeled antimouse antibodies can be generated by a variety of techniques known to those in the art. The labelled anti-mouse antibodies will bind to the antibody component of the sera that was operative in the assay and will thereby reveal the specific localization of the function performed by the gene product as revealed by the assay. Thus, the label may indicate that the antibody component of the sera is bound to a cell surface, is present intracellularly or extracellularly, or is affixed to a particular cell structure. These findings provide useful information about the localization of the gene product and indicate the necessary pathway to develop a diagnostic or therapeutic product.

All of the information yielded by the results of the assays provide a wealth of information about the relationship between the gene sequence, the gene product, and the biological process or physiologic function tested by the assay. In addition to pinpointing a specific gene sequence expressing a gene product involved in the process or function tested by the immunoassay, where the results show that binding of an antibody controls a pathology, for example, by killing tumor cells, the scientist learns how, when, and where the gene product reacts at the point in the biological process or physiological condition function that matters most, i.e., the point at which the reaction by the gene product is outcome determinative of the biological process or physiological function of interest. The basic methodology of this invention is a departure from prior art methodology because this invention has the capability to identify the gene product upon which the biological process or physiological condition is result dependent in the context of the assay employed. Therefore, this invention separates gene products that are merely coincident to a pathology from those that actually cause, control, or are capable of arresting or reversing the pathology of interest.

As noted above, once a specific gene product is identified by the assay(s), the corresponding gene sequence is examined in detail to determine the nature and identity of the product encoded by the gene sequence. When the gene product is identified, a therapeutic compound can be developed by conventional means based on this information. For example, the basic methodology of the invention may identify (or discover) a gene product that alleviates the effects of a disease or prevents the initiation or progression of a disease. In this case, the therapeutic product may be the gene product itself, e.g., derivative of the gene product. In such a case, using the information obtained by the basic methodology of the invention, the gene product can be produced in large quantities by conventional synthetic or recombinant methods, such as eukaryotic or prokaryotic expression systems.

Alternatively, the information obtained from the basic methodology of the invention may dictate that an antibody to the gene product may be the valuable therapeutic agent. In such a case, an antibody compatible for administration to a human may be obtained by known methods (Diaz Blasco, J., and P. Robledo Andres. 1995. [Advances in the topical steroid treatment of inflammatory bowel diseases]. Rev Esp Enferm Dig 87, no. 11:802-7; Amosova, E. N., and L. L. Sidorova. 1994. [The diagnostic significance of creatine phosphokinase antibodies in the cardiac muscle in non-coronarogenic myocardial diseases]. Vrach Delo, no. 1:37-9). For example, recombinant chimeric antibodies may also be produced by known genetic engineering techniques and produced by expression in prokaryotic or eukaryotic expression systems. Human antibodies may also be obtained by immunization of transgenic mice having selected DNA segments from a human immunoglobulin heavy and light chain gene locus incorporated into its germline DNA. See U.S. Pat. No. 5,633,425 which is incorporated herein by reference.

Additionally, small molecules may be obtained or designed to impede or enhance the function of a gene product. For example, the basic methodology of the invention may discover an important enzyme involved in a disease process, and inhibition of the enzymatic activity may provide a therapeutic effect. For example, angiotensin-covering enzyme (ACE) is an enzyme that transforms angiotensin I to angiotensin II (Luscher, T. F., R. R. Wenzel, P. Moreau, and H. Takase. 1995. Vascular protective effects of ACE inhibitors and calcium antagonists: theoretical basis for a combination therapy in hypertension and other cardiovascular diseases. Cardiovasc Drugs Ther 9 Suppl 3:509-23), which is involved in hypertension. Therefore, the basic methodology of this invention may dictate that the therapeutic intervention could be the best achieved by the design of small molecule (an enzyme inhibitor, which, in this case, inhibits ACE) to block the conversion from angiotensin I to angiotensin beta, ultimately leading to a decrease of hypertension (Luscher, T. F., R. R. Wenzel, P. Moreau, and H. Takase. 1995. Vascular protective effects of ACE inhibitors and calcium antagonists: theoretical basis for a combination therapy in hypertension and other cardiovascular diseases. Cardiovasc Drugs Ther 9 Suppl 3:509-23). A distinct advantage of the present invention is provided by the ability to rely on many existing techniques in the field of immunology, molecular biology, genetic engineering (Maniatis, T., Fritsch, E. F. and Sambrook, T. (1982). Molecular Cloning: A Laboratory Manual. Cold Spring Harbor Lab, Cold Spring Harbor, N.Y.), etc. while substantially enhancing their utility for curing disease by establishing a direct one-to-one relationship between a gene sequence and gene product and the cause and effect of that gene sequence or gene product in a selected biological process or physiological function.

Once a particular gene product is identified and the gene sequence isolated, additional information can be discovered by more focused analysis of the sequence and the resulting gene product. For example, a series of different antibodies can be raised against different portions of the identified gene product coded by the gene sequence to determine the critical portion of the gene product, i.e., which epitopes are operative in the binding reaction that is significant in the assay. Likewise, the discovery that the binding of an antibody with a cell component, such as a cell surface marker, controls a pathology provides the ability to select additional assays or tests to elucidate how and why the interaction between the antibody and the cell component exhibits the result displayed by the assay. Thus, additional analyses may contribute to the totality of information derived from performing the basic methodology of the invention to enhance the ability to diagnose and to intervene, dispositively, in a selected biological process or physiological function.

As noted above, one primary aspect of the invention is the discovery of novel genes, gene sequences, and novel gene products, which may literally be novel, or which may be previously known in the art, but whose specific function is underestimated, unrecognized, or poorly understood (Chan, V., B. Yip, T. P. Chan, and T. K. Chan. 1995. Uses of dinucleotide/trinucleotide repeats in the diagnosis of genetic diseases. Southeast Asian J Trop Med Public Health 26 Suppl 1:179-81; Tzourio, C., J. Maccario, and A. Alperovitch. 1995. Estimation of the proportion of genetic cases in late-onset diseases: an application to Alzheimer's disease. Stat Med 14, no. 12:1353-63; Shimron-Abarbanell, D., H. Harms, J. Erdmann, M. Albus, W. Maier, M. Rietschel, J. Korner, B. Weigelt, E. Franzek, T. Sander, M. Knapp, P. Propping, and M. M. Nothen. 1996. Systematic screening for mutations in the human serotonin 1F receptor gene in patients with bipolar affective disorder and schizophrenia. Am J Med Genet 67, no. 2:225-8). Additionally, the gene sequence may previously have been isolated, described, or characterized, however, no specific function for the sequence has been described or identified. Moreover, because the function determinative assay is preferably performed on both normal and pathological cells and/or tissue in parallel, the basic methodology of the invention distinguishes between forms of a particular gene product, or among multiple versions of the same gene product, e.g., native, truncated, or mutated forms of a gene product.

A further embodiment of the invention is the ability to identify or discover genes or gene sequences that are up- or down-regulated as part of a particular pathology. Any quantitative assay, such as immunofluorescence, ELISA sandwich, performed in parallel on both a normal cell type and its pathological counterpart can reveal the up- or down-regulation of specified gene sequences. This capability is particularly important in the study of diseases causing the up- or down-regulation of large numbers of genes because substantial difficulty in identifying genes or gene sequences whose expression product is significant in a particular pathology.

Therefore, the present invention takes advantage of (a) the efficiency and the specificity of the mammalian immune response (Tonegawa, S. 1993. The Nobel Lectures in Immunology. The Nobel Prize for Physiology or Medicine, 1987. Somatic generation of immune diversity [classical article]. Scand J Immunol 38, no. 4:303-19; Winter. G, and M. C. 1991. Man-made antibodies. Nature 349:293-299; Kohler and Milstein., 1975. Nature, 256: 495-497) against a particular antigen in any form, (b) the ability to use the functional components of the immune system of a mammal with a defined genetic background to mount a distinct and predictable immune response against an exogenous antigen to the mammal, (c) the availability, and the possibility to generate, desired cDNA libraries, preferably derived from human DNA and the need to discover the function of new genes, their corresponding products, and respectively their antagonists, and (d) the existing capability to identify or develop diagnostic or therapeutic compounds once knowledge of the specific function of a gene sequence or gene product relative to a pathologic condition is discovered. As noted above, once the particular gene sequence is isolated, the strategy for developing a therapeutic product will depend on the nature and identity of the compound that is encoded by the gene sequence(s).

### DETAILED DESCRIPTION OF PREFERRED EMBODIMENTS OF THE INVENTION

The term “gene” is frequently used in the art to describe a discrete DNA segment having a known biological function. The term “gene sequence” is used herein to describe a polynucleotide sequence that codes for a gene product. The term “gene sequence” is not used to define a different entity from a “gene,” but is used to indicate that the polynucleotide sequence(s) analyzed by the methodology of the invention will often have no known function. Thus, a “gene sequence” described herein may be comprised of a portion of a gene, an entire gene, an entire gene and an additional polynucleotide, or any fragments thereof.

The term “gene product” is used to describe the partial or total polypeptide resulting from the expression of a “gene sequence,” and may include fragments, derivatives, mutations, or isoforms of the polypeptide encoded by the polynucleotide. The gene product may also be glycosylated, phosphorylated, or conjugated with a lipid.

The term “gene product tag” (GPT) is used to describe an antibody or an immunoglobulin specific to a “gene product.”

The terms “function determinative assay” are used herein to describe one or more assays, including at least one immunoassay using the antibody (GPT) specific to the gene product, that yield information about a biological process or physiological phenomenon.

The mammalian immune system consists of several components that interact to produce an immune response to an antigen. This invention takes advantage of the ability of the mammalian immune system to readily generate sera containing antibodies that are highly specific to an antigen. The cells of the immune system that generate an immune response are quite diverse and undergo a process of maturation and differentiation prior to acquiring a cytotoxic function or the capability to produce specific antibodies against a wide variety of immunogens, varying from viruses to other components. The best characterized and most important parts of the mammalian immune system are the humoral and cellular (cytolytic) branches. The humoral component consists of a class of soluble immunoglobulin molecules found in the serum (antibodies) that are characterized by enormous diversity as well as specificity. These antibodies are synthesized by differentiated B-cells, which are derived from the bone marrow precursors. B-cell immunity is characterized most simply by the accumulation of specific antibodies to specific foreign molecules or antigens. The practical advantage of the use of an available mammalian immune system is to provide a source for unique and powerful reagents used in the basic methodology of this invention.

Antibodies are complex proteins that are the effectors of humoral immunity and are secreted into the body fluid of a mammal. If the immune system is exposed to an antigen, a fraction of B-cells will progress through a maturation process involving the rearrangement of the immunoglobulin gene and ultimately leading to the manufacture of specific antibodies that recognize and specifically bind to the antigen. Antibodies can bind and inactivate antigen directly (neutralizing antibodies) or activate other cells of the immune system to destroy the antigen. Alternatively, the binding of an antibody, on a cell receptor for example, may trigger a cascade of events in the cell and lead to cell proliferation, growth, differentiation and even death. The cellular system, in contrast, relies on special cells which recognize and kill other cells which are producing foreign antigens or presenting features that dictate to the immune system to remove them. The basic functional division between the humoral and cellular components of the immune system reflects at least two different strategies of immune defenses and probably others that are unknown at the present time. Humoral immunity is mainly directed at antigens which are exogenous to the animal, whereas the cellular system responds to antigens which are actively synthesized within the animal such as invasive viruses or parasites.

The cellular component of immunity relies most heavily on lymphocytes dependent on the thymus for their development and maturation (T cells). Cellular immune recognition is mediated by a special class of lymphoid cells, the cytotoxic T cells. These cells do not recognize whole antigens but instead they respond to degraded peptide fragments thereof which appear on the surface of the target cell bound to proteins called class I major histocompatibility complex (MHC) molecules. Essentially all nucleated cells have class I molecules. It is believed that proteins produced within the cell are continually degraded to peptides as part of normal cellular metabolism. These fragments are bound to the MHC molecules and are transported to the cell surface. Thus, the cellular immune system is constantly monitoring the spectra of proteins produced in all cells in the body and is poised to eliminate any cells producing foreign antigens. In addition to these features of the immune system, once the immune system has been exposed to a foreign antigen, a “fingerprint” remains in the cellular memory at the immune system. In this case, if the immune system recognizes the same antigen in the future, the immune reaction is more effective and more rapid. This capacity of the immune system has been used effectively in preparing vaccines.

Vaccination is the process of preparing an animal to respond to an antigen. Vaccination is more complex than immune recognition and involves not only beta cells and cytotoxic T cells but other types of lymphoid cells as well. During vaccination, cells which recognize the antigen (beta cells or cytotoxic T cells) are clonally expanded. In addition, the population of auxiliary cells (helper T cells) specific for the antigen also increase. Vaccination also involves specialized antigen presenting cells which can process the antigen and display it in a form which can stimulate one of the two pathways or both, and presumably others which are poorly understood at the present time.

The procedure of vaccination has changed very little since Louis Pasteur. A foreign antigen is introduced into an animal where it activates specific beta cells by binding to surface immunoglobulins and is taken up by antigen presenting and processing cells, degraded in specialized cellular compartments, and is presented to helper T cells in small fragments by the major histocompatibility complex (MHC) of class II. The interaction of these MHC class II molecules with the peptide and the T cell receptor of a helper T cell results in the production of an active humoral response. Thus, at least two different and distinct pathways of antigen processing and presentation produce exogenous antigens bound to class II MHC molecules where they can stimulate T helper cells, as well as endogenous proteins degraded and bound to class I MHC molecules and recognized by the cytotoxic class of T cells.

The humoral system protects a vaccinated individual from subsequent challenge from a pathogen by making specific binding antibodies to the pathogen to prevent the spread of an intracellular infection if the pathogen goes through an extracellular phase during its life cycle; however, it can do relatively little to eliminate intracellular pathogens. Cytotoxic immunity complements the humoral system by eliminating the infected cells. Thus, effective vaccination activates both types of immunity.

B-cell immunity, characterized most simply by the accumulation of specific antibodies to specific foreign molecules or antigens, has been the subject of intense biochemical and cellular study for almost a century. The practical advantage provided by this invention of an immunological approach to the study of gene products coded by the gene sequences is that, when properly exploited using the techniques described herein, the mammalian immune system provides antibodies that are unique and powerful reagents that are highly useful in function determinative assays. The past fifty years have witnessed the development of precise methods for the measurement of antibodies and their binding activity, an understanding of the molecular genetics of antibody molecules, which depends in large part on a set of novel reactions including the class-switch rearrangement and somatic mutation of immunoglobulin genes, the determination of the three-dimensional structure of a number of antibody fragments as well as of several antibody-antigen complexes, and the discovery and refinement of methods (Kohler and Milstein., 1975. Nature, 256: 495-497) for isolating and large-scale manufacturing of monoclonal antibodies (U.S. Pat. No. 4,946,778).

Over the years, the growing use of antibodies in all fields, and tremendous range of applications for specific antibodies, has also led to the manipulation of the antibody genes, either to alter the constant (C) region with which the variable (V) regions are associated, or to introduce designed changes in the antibody combining site. Contemporary PCR methods make it feasible to clone the antibody V region genes encoding both the heavy and light chains of the hybridoma, to ligate these into plasmid expression vectors encoding constant regions of human or other species, and to express them after transfection in myeloma cell lines capable of high-level production (Winter. G, and M. C. 1991. Man-made antibodies. Nature 349:293-299.52; Kohler and Milstein., 1975. Nature, 256: 495-497).

One particularly attractive aspect of the invention is the ability to discover that a particular gene product plays an important role in a disease by using specific antibodies to the gene product (GPTs) that react in a live cell of a mammal. The interaction between such a gene product and a specific antibody to the gene product can affect several biological processes such as growth inhibition, cell death, cellular differentiation, blockage of a receptor, blockage of infection by a pathogen, modulation of the production and secretion of hormones, and modulation of the production and secretion of lymphokines, to name a few. These important physiological conditions or phenomenons are just a few examples among many others that can be affected by the binding of a specific antibody (GPT) to the gene product in a living cell of a mammal. As will be readily appreciated, as a consequence of the binding of the antibody to the corresponding gene product, the practice of the basic methodology will also allow the de novo discovery of gene sequences, whether partial or complete, coding for corresponding gene products.

In addition to the basic methodology of the invention described herein, the present invention provides sufficient information to obtain antibodies to a gene product that are useful in virtually any therapeutic or diagnostic application where the function determinative assay indicates that binding of the gene product affects a biological process or physiological condition, most often a pathology or disease afflicting humans.

Antibodies Identified by the Methodology of the Invention as a Therapeutic or Diagnostic Substance

The basic methodology of the invention may identify a gene product where the binding of the gene product and its specific antibody results in killing cells in a tumor and thereby suggests a therapeutic strategy to provide a measurable reduction in the growth of a tumor in a patient. Consequently, substantially the same antibody can be used as a therapeutic agent against this type of tumor in vivo and may exert a therapeutic effect by any mechanism, e.g., antibodies that recognize cancer cells may have cytotoxic effects, may interfere with compounds or pathways necessary for tumor cell growth, or may initiate apoptosis. Additionally, even where tumor growth itself is not measurably altered, this invention identifies antibodies that may trigger, or interact with, substances or receptors specifically produced or present in specific cancer cell lines. Antibodies to gene products identified pursuant to the invention may also have additional utility beyond their use as therapeutic agents, i.e., compounds that directly assert a therapeutic effect. For a given gene product, antibodies obtained pursuant to the invention may also be used to purify the recognized gene product, to perform drug discovery as a therapy, or to develop diagnostic products to track the development, initiation, or progression of a disease. Moreover, the antibody can be used also to discover an antagonist that would exert an anti-tumor activity. Several such diagnostic and therapeutic applications utilizing antibodies to gene products identified pursuant to the basic methodology described herein are within the skill of those in the art once a gene sequence or gene product has been identified based on the teachings herein.

Antibodies to the gene product identified by the invention may be polyclonal, monoclonal, chimeric, single chain, Fab fragments, or the product of a Fab expression library. Various procedures known in the art may be used for the production of any of these types of antibodies. For the preparation of monoclonal antibodies, any technique which provides antibodies produced by continuous cell line cultures can be used. For examples, the hybridoma technique (Kohler and Milstein, 1975, Nature, 256:495-497), the trioma technique, the human B-cell hybridoma technique (Kozbor et al., 1983, Immunology Today 4:72), and the EBV-hybridoma technique (Cole et al., 1985, human monoclonal antibodies and cancer therapy, Alan R. Liss, Inc., pp. 77-96), each produce suitable monoclonal antibodies. Techniques described for the production of single chain antibodies (U.S. Pat. No. 4,946,778) can also readily be adapted to produce single chain antibodies to immunogenic gene products of this invention. Phage display techniques as described in U.S. Pat. Nos. (4,797,363 Teodorescu et al., 4,987,073 Berman et al., 5,223,409 Ladner et al., and 5,338,665 Schatz et al.) can also be used to produce antibodies against any gene product or fragment thereof and/or to mimic the interaction between an antibody and a gene product.

Therefore, a preferred embodiment of the present invention is the use of a gene product, identified by the methodology of the invention, to obtain a substance for therapeutic intervention that acts through the antibody action on the gene product or any of its derivatives. For example, the practice of the basic methodology of the invention may reveal specific markers associated with pathological disorders such as cancer, autoimmune disease, neuro-regenerative disease or other diseases. In such a situation, the administration of an antibody specific to the marker-associated disease, obtained by any variety of methods in molecular immunology known to those skilled in this art, will bind specifically to a target cell (cancer cells, infected cells or otherwise) in the mammal and which exerts a therapeutic effect by any of several pathways, for example, the fixation of complement, which results in lysis, or phagocytosis of the cell by various effector cells (a cell of the immune system capable of responding, often by killing a target cell) such as natural killer (NK) cells, neutrophils, monocytes, or macrophages. The basic methodology may also reveal that an antibody binding reaction is occurring in a particular disease with compounds such as critical hormones, growth factors, interleukins, or other regulatory molecules or their receptors that control growth of these cells.

In another embodiment of the present invention, antibodies to specific markers associated with tumors are used in imaging or diagnosis, particularly radioimmunodiagnosis of solid tumors. In such a case, a specific antibody may be labelled with a radioisotope such as iodine 131 (¹³¹I) and injected into the patient. The labelled ¹³¹I antibody circulates in the whole body and binds to its specific target. Radioimaging will show the localization of the solid tumor and its size.

Another therapeutic method of the present invention, is the use of anti-idiotypic antibodies based on specific markers associated with tumors or inflammatory disease. Idiotypes are antigenic markers of the variable region of immunoglobulin molecules and the set of idiotypes on an antibody molecule constitutes an antibody's idiotype. Antibodies that recognize idiotypes are termed anti-idiotypic antibodies. An example of this type is documented by B-cell myeloma: the B-cell malignancies are clonal diseases that express unique surface immunoglobulin and the immunoglobulin idiotype represents a particular tumor specific antigen. As a result, binding of an anti-idiotypic antibody to malignant B-cells triggers an inflammatory response that kills only these tumor B-cells.

DNA and/or RNA Identified by the Methodology of the Invention as Therapeutic or Diagnostic Substance

A polynucleotide sequence, whether a partial or complete gene sequence, may have a direct therapeutic effect as a nucleotide sequence of DNA, or as its corresponding mRNA, or directly as the gene product of the administered gene sequence.

The polynucleotide sequences identified by the methodology of the invention are comprised of DNA or RNA sequences having a therapeutic effect after being taken up by a cell, or may be used solely as immunogens to produce specific antibodies capable of recognizing specific gene products in the cell of the mammal. Examples of polynucleotides that may be employed as therapeutic agents are anti-sense DNA and RNA, DNA coding for an anti-sense RNA, or DNA coding for tRNA or rRNA to replace defective or deficient endogenous molecules (Jaksch, M., S. Hofmann, P. Kaufhold, B. Obermaier-Kusser, S. Zierz, and K. D. Gerbitz. 1996. A novel combination of mitochondrial tRNA and ND1 gene mutations in a syndrome with MELAS, cardiomyopathy, and diabetes mellitus. Hum Mutat 7, no. 4:358-60; Nakanishi, M., G. R. Adami, R. S. Robetorye, A. Noda, S. F. Venable, D. Dimitrov, O. M. Pereira-Smith, and J. R. Smith. 1995. Exit from GO and entry into the cell cycle of cells expressing p21Sdi1 antisense RNA. Proc Natl Acad Sci U S A 92, no. 10:4352-6). The polynucleotides of the invention can also code for therapeutic polypeptides. The gene product may be any translation product of a polynucleotide regardless of size, and whether or not glycosylated in any other chemical or biochemical complexes. The gene product encoded by therapeutic polynucleotides include, as primary examples, any gene product that can compensate for defective genes in a mammal or any gene product that acts through physiological pathways to elicity a toxic effect to limit or remove harmful cells from the body.

Therapeutic polynucleotides provided by the invention can also code for immunity-conferring gene products, which can act as endogenous immunogens to provoke a humoral or cellular response, or both. The polynucleotides employed according to the present invention can also code for an antibody. In this regard, the term “antibody” encompasses whole immunoglobulin of any class, chimeric antibodies and hybrid antibodies with dual or multiple antigen or epitope specificities, and fragments, such as F(ab)₂, Fab′, Fab and the like, including hybrid fragments. Also included within the meaning of “antibody” are conjugates of such fragments, and so-called antigen binding proteins (single chain antibodies) as described, for example, in U.S. Pat. No. 4,704,692.

Thus, a therapeutic polynucleotide coding for the variable and/or constant regions of an antibody can be introduced, in accordance with the present invention, to enable the patient to produce a selected antibody in situ. For illustrative methodology relating to obtaining antibody-encoding polynucleotides, see Ward et al. Nature, 341:544-546 (1989); Gillies et al., Biotechnol. 7:799-804 (1989); and Nakatani et al., loc. cit., 805-810 (1989). Such an antibody may exert a therapeutic effect, for example, by binding a surface antigen associated with a pathogen. Alternatively, the encoded antibodies can be anti-idiotypic antibodies (antibodies that bind other antibodies) as described, for example, in U.S. Pat. No. 4,699,880. Such anti-idiotypic antibodies could bind endogenous or foreign antibodies in a treated individual, thereby to ameliorate or prevent pathological conditions associated with an immune response, e.g., in the context of an autoimmune disease.

Therapeutic polynucleotide sequences may also code for regulatory proteins that control the expression of selected gene products. Such regulatory proteins can act by binding to genomic DNA to regulate transcription, or can function by binding to messenger RNA to increase or decrease RNA stability or translation efficiency.

The polynucleotide material delivered in vivo can take any number of forms. Plasmids containing genes coding for a large number of physiologically active gene products that function as antigens or immunogens can be readily obtained by those of skill in the art.

When a polynucleotide is administered as a therapeutic agent, the cell into which the polynucleotide is introduced can be a dividing or non-dividing cell in the tissue of the mammal. In preferred embodiments, the polynucleotide is introduced into muscle tissue; in other embodiments the polynucleotide is incorporated into tissues of skin, brain, lung, liver, spleen or blood or other tissues of the mammal. The preparation is injected by a variety of routes, which may be intradermally, transdermally, subdermally, intraperitoneally, intravenously, or it may be placed within cavities of the body or inhaled (Bourne, N., G. N. Milligan, M. R. Schleiss, D. I. Bernstein, and L. R. Stanberry. 1996. DNA immunization confers protective immunity on mice challenged intravaginally with herpes simplex virus type 2. Vaccine, 14 no. 13:1230-4; Lawson, C. M., J. R. Bennink, N. P. Restifo, J. W. Yewdell, and B. R. Murphy. 1994. Primary pulmonary cytotoxic T lymphocytes induced by immunization with a vaccinia virus recombinant expressing influenza A virus nucleoprotein peptide do not protect mice against challenge. J Virol 68, no. 6:3505-11; Vadolas, J., J. K. Davies, P. J. Wright, and R. A. Strugnell. 1995. Intranasal immunization with liposomes induces strong mucosal immune responses in mice. Eur J Immunol 25, no. 4:969-75; Winegar, R. A., J. A. Monforte, K. D. Suing, K. G. O'Loughlin, C. J. Rudd, and J. T. Macgregor. 1996. Determination of tissue distribution of an intramuscular plasmid vaccine using PCR and in situ DNA hybridization. Hum Gene Ther 7 no. 17:2185-94; Yokoyama, M., J. Zhang, and J. L. Whitton. 1996. DNA immunization: effects of vehicle and route of administration on the induction of protective antiviral immunity. FEMS Immunol Med Microbiol 14, no. 4:221-30). In a preferred embodiment, the polynucleotide is injected intramuscularly. When the polynucleotide is DNA, it can also be a DNA sequence which is itself non-replicating, but is inserted into a plasmid, and the plasmid further comprises a replicator. The DNA may be a sequence engineered so as not to integrate into the host cell genome. The polynucleotide sequences may code for a gene product which is either contained within the cells or secreted therefrom, or may comprise a sequence which directs the secretion of the gene product.

For example, C-myc an oncogene gene product is overexpressed in glioblastoma, the most common form of brain tumor. Other genes overexpressed in other tumors or inflammatory diseases are well-known in the literature. In these cases, one therapeutic intervention will be to decrease the over-expression of these genes at the transcription level using anti-sense polynucleotide sequences to the targeted gene. Where the use of antisense sequences, whether RNA or DNA and/or their corresponding gene products, are the preferred means of therapeutic intervention, polynucleotide sequences or the corresponding gene products or derivatives are administered in different forms according to the tissue, the type of disease to be controlled, and the necessity to control the pathology for varying lengths of time. In addition, anti-sense polynucleotide sequences can also be administered at the same time as other pharmaceutical substances, as dictated by each clinical indication.

In many instances, it is preferred that the polynucleotide is translated for a limited period of time so that the gene product delivery is transitory. The gene product may advantageously be comprised of a therapeutic enzyme, hormone, lymphokine, receptor, regulatory protein, such as a growth factor or other regulatory agent, or any other protein or gene product (regardless of size and nature) that one desires to deliver to a cell in a living vertebrate and for which corresponding DNA or mRNA can be obtained.

Gene Products Identified by the Methodology of the Invention, and Their Derivatives, as Therapeutic Substances

The present invention encompasses the therapeutic use of gene products identified by the basic methodology of the invention and, preferably, the use of gene sequences coding for the gene products to produce therapeutic agents for a particular pathological condition. The following are merely illustrative examples of a larger number of pathologies where the use of the basic methodology allows the discovery of genes which code for gene products that have therapeutic value.

Discovery of New Growth Hormones

In this example, the expression of a gene sequence to yield a gene product that controls physiological growth (Cohen, S. B., P. D. Katsikis, C. Q. Chu, H. Thomssen, L. M. Webb, R. N. Maini, M. Londei, and M. Feldmann. 1995. High level of interleukin-10 production by the activated T cell population within the rheumatoid synovial membrane. Arthritis Rheum 38, no. 7:946-52; Du, X. X., D. Scott, Z. X. Yang, R. Cooper, X. L. Xiao, and D. A. Williams. 1995. Interleukin-11 stimulates multilineage progenitors, but not stem cells, in murine and human long-term marrow cultures. Blood 86, no. 1:128-34; Berg, D. J., M. W. Leach, R. Kuhn, K. Rajewsky, W. Muller, N. J. Davidson, and D. Rennick. 1995. Interleukin 10 but not interleukin 4 is a natural suppressant of cutaneous inflammatory responses. J Exp Med 182, no. 1:99-108; Cacabelos, R., X. A. Alvarez, L. Fernandez-Novoa, A. Franco, R. Mangues, A. Pellicer, and T. Nishimura. 1994. Brain interleukin-1 beta in Alzheimer's disease and vascular dementia. Methods Find Exp Clin Pharmacol 16, no. 2:141-51), is identified based on the methodology of the invention. More specifically, the binding of an antibody (GPT), generated according to the practice of this invention, to a candriocyte or neuronal cell line results in the interruption of the growth of these cells. This result demonstrates that the expression of the identified gene product is crucial for the growth of these cells. Thus, the expression of this gene product in candriocyte cells, or neuronal cells is shown to make them grow more rapidly. In this situation, the gene product or derivatives thereof can be used to control a variety of pathological conditions in a mammal that affect the bone or neuronal cells. In clinical application, such growth hormones are useful in hip fractures and to cause neuronal regeneration. Similar experiments using the basic methodology of this invention, and directed specifically to hematopoietic stem cells can identify growth factors, such as erythropoietin, and other factors that act directly on the growth or differentiation of hematopoietic stem cells and which will be useful in controlling many pathological conditions. Similarly, growth factors for skin regeneration or stem cell differentiation can be identified.

Therapeutic Gene Products: Hormones, Such as Somatostatin, Insulin, Glucagon, are Involved in the Endocrine System Related to Alimentation and Metabolic Fuel Integration are Useful in a Variety of Therapeutic Indications

The invention may identify a particular hormone that is desirable to treat a given pathological condition. Once administered, cells of the tissue express the gene product. In this case, the gene product is therapeutic and the administration of the gene product to a mammal in need of therapy, is provided directly by the gene product which has a therapeutic effect (Hurford, R. K., Jr., G. Dranoff, R. C. Mulligan, and R. I. Tepper. 1995. Gene therapy of metastatic cancer by in vivo retroviral gene targeting. Nat Genet 10, no. 4:430-5; van Gelder, T., A. H. Mulder, A. H. Balk, B. Mochtar, C. J. Hesse, C. C. Baan, L. M. Vaessen, and W. Weimar. 1995. Intragraft monitoring of rejection after prophylactic treatment with monoclonal anti-interleukin-2 receptor antibody (BT563) in heart transplant recipients. J Heart Lung Transplant 14, no. 2:346-50; Belkowski, S. M., C. Alicea, T. K. Eisenstein, M. W. Adler, and T. J. Rogers. 1995. Inhibition of interleukin-1 and tumor necrosis factor-alpha synthesis following treatment of macrophages with the kappa opioid agonist U50, 488H. J Pharmacol Exp Ther 273, no. 3:1491-6). In another embodiment, a pharmaceutical product, comprised of the naked polynucleotide that operatively codes for the biologically active gene product can be administered as a therapeutic agent, i.e., in solution in a physiologically acceptable injectable carrier and suitable for introduction interstitially into a tissue.

In another embodiment, the gene product encoded by the administered polynucleotide may have a therapeutic effect through an additional pathway, such as the compensation of a genetic deficiency for a critical gene product, where the deficiency causes the disease. The method of polynucleotide delivery, and whether the polynucleotide is designed to integrate into the mammalian genome or not, will depend on the type of disease to be corrected and the severity.

The polynucleotide may also be introduced into muscle cells, for example, where the polynucleotide codes for the dystrophin gene product, the polynucleotide acts as a pharmaceutical agent for the treatment of muscular dystrophy (Cox, G. A., N. M. Cole, K. Matsumura, S. F. Phelps, S. D. Hauschka, K. P. Campbell, J. A. Faulkner, and J. S. Chamberlain. 1993. Overexpression of dystrophin in transgenic mdx mice eliminates dystrophic symptoms without toxicity [see comments]. Nature 364, no. 6439:725-9; Fritz, J. D., I. Danko, S. L. Roberds, K. P. Campbell, J. S. Latendresse, and J. A. Wolff. 1995. Expression of deletion-containing dystrophins in mdx muscle: implications for gene therapy and dystrophin function. Pediatr Res 37, no. 6:693-700).

In another example, the gene product is crucial to the operation of the liver. In this case, hepatic cells can be removed by hepatoectomy, treated to uptake the polynucleotide that codes for the missing enzyme and reinjected back into the liver again. For example, the hypercholesteremia is due to a deficiency of low density lipid receptor (LDL receptor). The operative introduction of a functional LDL receptor alleviates hypercholesteremia.

The discovery of critical gene sequences, gene products, and their physiological function related to pathological conditions also allows the discovery of a missing genes where the normal function of the gene is critical for maintaining normal physiological condition, such as a normal immune response (Blaese, R. M., K. W. Culver, A. D. Miller, C. S. Carter, T. Fleisher, M. Clerici, G. Shearer, L. Chang, Y. Chiang, P. Tolstoshev, and et al. 1995. T lymphocyte-directed gene therapy for ADA-SCID: initial trial results after 4 years. Science 270, no. 5235:475-80), including the avoidance of autoimmune disorders as described in more detail below. In such an example, a therapeutic intervention may transform stem cells from bone marrow with a polynucleotide sequence (Bordignon, C., L. D. Notarangelo, N. Nobili, G. Ferrari, G. Casorati, P. Panina, E. Mazzolari, D. Maggioni, C. Rossi, P. Servida, and et al. 1995. Gene therapy in peripheral blood lymphocytes and bone marrow for ADA-immunodeficient patients. Science 270, no. 5235:470-5). In this embodiment, the bone marrow cells may be transfected in vitro with a polynucleotide, the polynucleotide incorporated into the cells, and the cells re-injected into the mammal. The polynucleotide may code for a secreted gene product by the cell, or a cell surface or intracellular component.

Gene Product Administered To Cause A Desired Immune Response

This invention also identifies gene sequences gene products that are used to confer immunity to a mammal by producing a desired immune response. In such cases, the gene products act as immunogens to provoke a humoral or cellular response, or both.

In embodiments wherein the cell expresses Class I major histocompatibility complex, and the immunogenic gene product is presented in the context of the Class I complex, the immune response may be cellular and comprises the production of cytotoxic T-cells. In one such embodiment, the immunogenic gene product is associated with a pathogen such as a virus, or other infectious microorganism and is presented in the context of Class I antigen complex. In this example, the gene product stimulates cytotoxic T-cells which are capable of destroying cells infected with the virus.

Within the same context, the gene product encoded by the corresponding gene sequence may be secreted outside the cell to act directly against the infectious pathogen, or may block its site of entrance on a cell, thereby preventing it from infecting the mammal. The gene product coded by the gene sequence may also directly act as a toxic agent against the pathogen or trigger the cells in the mammal to destroy the infectious agent by a variety of known or unknown physiological mechanisms.

In accordance with another aspect of the present invention, the practice of this methodology identifies an immunogenic gene product that successfully immunizes a mammal against a particular pathological condition. In such a case, the mammal is immunized by the steps of obtaining a preparation comprising an expressible polynucleotide coding for the immunogenic gene product, introducing the preparation into a mammal wherein the gene product of the polynucleotide is formed in vivo by a cell of the mammal, and eliciting an immune response against the immunogen thereby resulting in the protection of the mammal against the pathological condition.

In another embodiment, where the immunogenic gene product is associated with a tumor, the gene product is presented in the context of Class I antigens and stimulates cytotoxic T cells that are capable of destroying tumor cells. An analagous strategy to destroy tumor cells may be designed to boost both humoral and cellular immune responses that translate into the production of both antibodies and cytotoxic T-cells. By this mechanism, strong and specific immunogological reaction is created that will destroy or reduce a tumor by either rejection or necrosis of tumoral cells.

The polynucleotide coding for an immunogenic gene product may be used to immunize a mammal and to provide a better delivery of the polynucleotide using a positively charged liposome. The liposome may be incorporated into a monocyte, a macrophage, or another cell, where an immunogenic gene product of the polynucleotide is formed. The gene product is processed and presented by the cell in the context of the major histocompatibility complex, thereby eliciting an immune response against the immunogen.

In any of the embodiments of the invention where a liposome may be advantageously used, for example, when a polynucleotide is associated with a liposome, suitable materials for forming liposomes, preferably cationic or positively charged liposomes, or other vesicles having membranes comprised of lipids are employed, see e.g., Unger U.S. Pat. No. 5,585,112 and references cited therein.

Identification of Therapeutic Applications Involving the Immune Response

In addition to providing an immunogen to stimulate an immune response, the basic methodology of the invention identifies other factors, i.e., co-factors, cell surface markers, etc., that create or prevent an immune response. For example, an immune response may involve autoreactive T-cells that induce disease in mammals. Autoimmune diseases including rheumatoid arthritis, multiple sclerosis, myasthenia gravis, and systemic lupus erythematosus (SLE) are thought to be caused by tissue destruction mediated by autoreactive T-cell clones. In the field of transplantation, graft rejection is also mediated by host T cell recognizing foreign determinants on the allograft, while graft-versus-host disease (GVHD) is mediated by mature T cells within the donor bone marrow. Pursuant to this invention, therapeutic agents comprised of any gene products, antibodies to gene product, or therapeutic polynucleotides, etc. may be identified and developed to alter the reactivity, differentiation or function of autoreactive T-cells and to treat autoimmune diseases and other immunological disorders.

These and other therapeutic and diagnostic application of the present invention, will be apparent to those skilled in the art from the following teachings of the basic methodology of the invention.

Basic Methodology

The practical application of the basic methodology is set forth in the following description and the references cited herein.

Polynucleotide Materials

The practice of the present invention requires selecting a polynucleotide (DNA and/or RNA) operatively coding for a partial or a complete gene product for immunization of a mammal. The polynucleotide operatively codes for a gene product and has all the genetic information necessary for its expression, such as a suitable promoter and the like. The polynucleotide may be a partial or complete sequence. These polynucleotide can be administered to a mammal by any method that delivers the gene product of the polynucleotide to the functional components of the immune system of a mammal, such as by injection into the interstitial space of tissues such as muscle or skin, introduction into the circulation or into body cavities or by inhalation or insufflation, or injection into any tissue of the mammal to produce an immune response. A single polynucleotide or combination of a certain number of selected polynucleotides is typically delivered to the animal with a pharmaceutically acceptable carrier. A preferred liquid carrier is partially or totally aqueous, and contains pyrogen-free sterile water and a physiologically compatible salt concentration. The pH of the carrier is suitably adjusted to physiological pH ranges.

The polynucleotides may or may not integrate in the recipient cell genome. The polynucleotides may be non-replicating DNA sequences, or may be genetically engineered to possess specific replicating elements to insure the maintenance and replication of the delivered polynucleotide to facilitate the continued expression of the gene product for extended periods.

The polynucleotide of the gene product to be delivered to the mammal can be in any of the following forms: a plasmid construct, separated from any construct, in any viral or proviral construct, or in any artificial chromosome. The polynucleotide can be delivered in any form, and in association with any additional chemical, mineral, or biochemical molecules, to increase the efficiency of delivery and stability of the polynucleotide and its uptake by the mammalian cells. The polynucleotide of the gene product may be replicated in a prokaryotic or eukaryotic system. The prokaryotic or eukaryotic system that facilitates the expression of the polynucleotide, in any of the forms previously described, may also be delivered to the mammal as a whole to be exposed to the immune system. In this case, the resulting antibodies in the mammal will be directed against the desired gene product of the polynucleotide, as well as against the gene product of the prokaryotic or eukaryotic system. Ultimately, these antibodies will be absorbed against the gene product of the prokaryotic or eukaryotic, prior to be used in the functional assays, described hereafter.

The polynucleotide sequences to be administered to the immune system of the mammal may be prepared from prokaryotic or eukaryotic expression cDNA libraries, referred to as cDNA, derived from particular tissues and/or cells of a mammal, preferably a human, and optionally derive from cells or tissues reflecting a specific pathology. The cDNA is preferably driven by a strong eukaryotic promoter such as RSV LTR, CMV, ACTIN or PGK and an appropriate regulatory element to achieve the highest possible expression of the administered cDNA in mammalian tissues. Preferably, libraries are enriched cDNA libraries from which “housekeeping” gene sequences are removed to increase the efficiency of discovery of specific gene sequences for the tissue from which the cDNA library was constructed (See, Maniatis, T., Fritsch, E. F. and Sambrook, T. (1982). Molecular Cloning: A Laboratory Manual. Cold Spring Harbor Lab, Cold Spring Harbor, N.Y..) The cDNA libraries may be purchased through a commercial vender (Clonetech CA, Strategene) or may be made by following standard methods as described in the Current Protocols In Molecular Biology John Wiley, edited by Frederick M. Ausubel et al., and according to methods for molecular biology described in Maniatis supra.

Enriched cDNA libraries may be made from tumor cells, neuronal cells, muscular cells, liver, skin, colon, intestine, testis, ovary, heart, muscle, pancreas, spleen, colon, bladder, brain, lung, prostate, endothelial cells, kidney, synovial membrane, bone marrow, stem cells, pancreas, blood cells (hematopoeitic cells), lymphoid cells, adrenal gland, adipose cells, candreocytes, testis, parathyroid, salivary gland, breast, ovary, placenta, uterus, gall bladder, and other mammalian cell types. These expressed cDNA libraries are plated pursuant to conventional methods, i.e., on a solid LB agar medium containing an antibiotic for selection of bacteria that contain a functional plasmid construct. The plasmid construct contains also a particular cDNA that could correspond to a full length or to a partial coding sequence of a given gene. The cDNA library is plated at an appropriate density for the growth of individual colonies. Individual colonies are grown in a sterile container in approximately 1 to 250 milliliters of an appropriate selective medium until growth saturation of the bacteria. Individual clones can also be grown in different containers such as in a multi-well setting. The bacteria are centrifuged, the supernatant discarded, and the pellet transferred to a multiwell plate to allow for an automated or a semi-automated purification of the polynucleotide. The purification of the polynucleotide is carried out following basically the procedure of alkaline lysis method as described in the Current Protocols In Molecular Biology, John Wiley, edited by. Frederick M. Ausubel et al., 1996, published by John Wiley & Sons, Inc.

Typically, if individual colonies were grown in 3 ml., for example, at the growth saturation curve, bacteria are centrifuged for 10 minutes at 4000 rpm, in Sorvall RC 5B in a swinging bucket rotor or similar system. The medium is discarded and the pellet is resuspended in 300 microliters or more of solution A (50 mM Tris HCl pH8, 10 mM EDTA disalt, 20% Sucrose and 25 microgram of Lysozyme and 4 micrograms of RNAase), transferred to multi-well plates, and kept at room temperature for 20 minutes. An equal volume of 300 microliters or more of lysing solution, Solution B (1% SDS and 0.2% of NaOH), is added to solution A containing the bacteria, and is mixed gently to allow a complete lysis of the bacteria. Finally, a volume of 300 microliters or more of neutralizing solution, Solution C (3 M potassium acetate) is added to the lysed bacteria and mixed thoroughly to allow the precipitation of the chromosomal DNA and proteins. Bacterial lysate are kept at 4° C. for 15 minutes. Then, multi-well plates are centrifuged in a Sorvall RC 5B in a swinging bucket rotor for 20 minutes at 4000 rpm. Supernatant is transferred to new multi-well plates and mixed to solution D (a volume of isopropanol equal to 0.7 of the total previous volume). Solution D is added to the supernatant harvested from the bacterial lysate to precipitate the polynucleotide samples. Samples are kept 20 minutes at −20° C. and then centrifuged for 15 minutes at 4000 rpm. The supernatant is discarded and the pellet solubilized in ±300 microliters of TE containing 2 micrograms of RNAase. Prior to its use, the DNA is filtered through G 25 Sephadex beads, under vacuum pressure. This procedure may also be coupled with a multi channel pipetter to automatically distribute the different solution involved in this process of polynucleotide preparation.

Alternatively, plasmid DNA is recovered by ethanol precipitation and purified though a commercial column for DNA purification (Quiagen). The solution is also purified using a double cesium chloride gradient to remove contaminant proteins and other components if required.

In addition to the above, other methods known in the art of molecular biology may be used to prepare polynucleotide templates to facilitate delivery of the gene product of the selected polynucleotide to the immune system of the mammal.

Preparation of Purified DNA Template

Generally, for a large preparation, the plasmid or DNA template is prepared as per Maniatis supra, using 2 CsCl spins to remove bacterial RNA. E. coli is grown up in 500 milliliter in ampicillin-containing LB medium. The cells are then pelleted by spinning at 5000 rpm for 10 minutes in a Sorvall RC-5 centrifuge (E. I. DuPont, Burbank, Calif. 91510), resuspended in 50 ml of a solution of 50 mM glucose, 25 mM Tris-Cl pH8.0, 10 mM EDTA, and 40 mg/ml of lysozyme. After incubation for 5 to 10 minutes, a volume of 50 ml of 0.2N NaOH 1% SDS is added, followed, after 10 minutes at 0 degrees C, with 50 ml of 3M potassium acetate. After 10 more minutes, the material is again centrifuged at 6000 rpm, and the supernatant removed gently and mixed into 0.7 volume of isopropanol mixed and stored at −20 degrees C. for 15 minutes. The solution containing the DNA is then centrifuged again at 10,000 rpm for 20 minutes, in an HB4 swinging bucket rotor apparatus (DuPont, supra) after which the supernatant is removed and the pellet washed in 70% EtOH and dried at room temperature. Next, the pellet is resuspended in 10 ml TE, followed by addition of 9.5 g CsCl and 400 μl of 5 mg/ml EtBr. The resulting material is placed in a quick seal tube and filled to the top with mineral oil. The tube is spun for 3.5 hours at 80,000 rpm using a VTi80 rotor and ultra-centrifuge (Beckman Instruments, Pasadena, Calif., 91051). The DNA migrates according to the density of the CsCl to form a distinguishable band. This band corresponds to the concentrated DNA solution and is removed. The DNA material is centrifuged again, making up the volume with 0.95 g CsCl/ml and 0.1 ml of 5 mg/ml of EtBr. The EtBr is then extracted with an equal volume of TE saturated NaCl and n-butanol after adding 3 volumes of TE to the DNA solution and discarding the upper phase until the upper phase is clear. Next, 2.5 vol. EtOH is added, and the material precipitated at minus 20 degrees C. for 2 hours. The resultant DNA precipitate is solubilized in TE, dialysed and used as a DNA template for further usage.

Semi-Automatic Polynucleotide Preparation

Individual clones are picked up from the plated cDNA library on LB-agar, using a yellow tip, and are transferred into a 96 well plate containing 150 μl of LB-medium with appropriate antibiotic and grown until saturation. The 96 well plate receives 100 μl of 80% glycerol in each well and is frozen at 80° C. Duplicates of the clones are grown in an appropriate similar setting and are harvested in multiwell plates, centrifuged, the medium discarded and the pellet resuspended into 1 ml of solution A (50 mM Tris/HCl pH8, 20% glucose, 1 mM EDTA) with gentle shaking using our orbital shaker. lml of solution B (0.2M NaOH, 1% SDS) is added to each single bacterial solution and kept for 10 minutes. Then 1 ml of solution C (3M Na-Acetate acetic acid at pH5.2) is added to each sample. Precipitates of bacterial and chromosomal DNA and precipitated proteins are removed and 2.5 ml of isopropanol is added to each sample. DNA templates are recovered by precipitation, resuspended in 300 μl of TE (10 mM Tris-HCl pH8, 1 mM EDTA), and transferred to 96 well plates until further usage.

DNA templates are then used for immunization, either individually or combined into pools of 2, 3, 4, 5, 6, 7, 8, 9, 10 clones or more, but preferably between 1 and 10 clones. However, combined DNA template pools used for immunization may contain greater than 10 clones and as much as 50 individual clones or any integral value or range of values therein, or more if necessary.

Large scale DNA Preparation

An enriched cDNA library is plated at the appropriate concentration to result in individual clones. Individual clones are picked-up and grown in approximately 5 ml LB medium in appropriate container such as 24 well or other well plates until complete growth saturation is achieved. Bacteria are harvested by centrifugation and used to prepare the polynucleotide template to deliver the gene product to the immune system of the mammal.

The methodology of the invention may use large scale samples of different clones from different cDNA libraries made from different tissues, and are preferentially maintained in an array, for example by using 96 well plates or in another similar setting for each clone that corresponds to a single coding polynucleotide. These clones are stored in this setting for any further characterization. For example, 100,000 individual polynucleotide clones from a brain library per se may be stored as individual clones in 96 well plates, yielding a total of approximately 1000 different plates.

The polynucleotide sequences may also be maintained for long term storage using approximately 2 μl of DNA template solution containing 2 μg of polynucleotide for each clone and overlaid on 2/2 mm² Whatman paper, on a sheet of sterile Whatman paper of 6250 mm². Thus, a library of 100,000 clones can be stored on approximately 80 Whatman papers of this nature.

Recombinant Gene Products Expressed in a Prokaryotic Expression System

The delivery of gene products of selected polynucleotides to the immune system of a mammal may be achieved by initially using a prokaryotic expression system. The cDNA libraries are constructed from cell lines or tissue using a plasmid construct such as Lambda gt10, Lambda gt11, Lambda ZAP, Lambda ZAP beta, Uni-ZAP XR vector from Stratagene Company/USA, as well as other commercial vectors. These vectors contain the LacZ gene that codes for the B-galactosidase (beta-galactosidase) enzyme. The LacZ nucleotide sequence contains the polycloning sites. Consequently, the insertion of a cDNA into the cloning site interrupts (a) the production of B-galactosidase functional enzyme, (b) and the formation of a fusion protein composed of the gene product coded by the cDNA and the rest of B-galactosidase. This chimeric gene product is referred to recombinant protein. The commercial availability of a specific antibodies against B-galactosidase which are attached to agarose beads allows the purification of the recombinant protein from E. Coli protein extract, by passing the E. Coli protein extract containing the recombinant protein through the anti-B-galactosidase column. E. Coli transformed with cDNA libraries are plated using standard method as described in the Current Protocol for Molecular Biology, by Frederick M. Ausubel, et al. Clones expressing recombinant protein are picked up individually from the original plate and grown into 250 ml overnight. The expression of recombinant protein may be induced by the addition of IPTG solution at 2 mM for six hours, if necessary, to induce a high level of production of the recombinant protein. E. Coli extracts are passed through the column of the anti-B-galactosidase beads to purify the desired gene product for immunization of the mammal (Maniatis, T., Fritsch, E. F. and Sambrook, T. (1982). Molecular Cloning: A Laboratory Manual. Cold Spring Harbor Lab, Cold Spring Harbor, N.Y.).

The production of recombinant gene products are produced as follows: Bacteria are grown and harvested by centrifugation and lysed in solution A (Tris-HCl pH7.5, 50 mM, EDTA 10 mM, Glucose 25%, and Triton×100 0.2%). Bacterial lysates are centrifuged at 27,000 rpm in a swinging rotor TST 28 for one hour to remove bacterial chromosomal DNA and precipitated proteins. Supernatant is then incubated with 3 ml of agarose beads conjugated with anti B-galactosidase antibody. Beads are washed twice with a solution of Tris-HCl pH7.5 25 mM, EDTA 2 mM, Triton×100 0.1%. Then, recombinant protein are removed from the beads using a glycine solution of 100 mM pH3. The polynucleotide of the gene product may also be translated and expressed in a prokaryotic or eukaryotic system. As described previously, the prokaryotic or eukaryotic system, expressing the desired gene product may be delivered to a mammal to be exposed to the immune system, in order to produce specific antibodies to the gene product. In this particular case, the antibodies in the mammal will be directed against the desired gene product of the polynucleotide, as well as against the gene product of the prokaryotic or eukaryotic system. Ultimately, these antibodies will be absorbed against the gene product of the prokaryotic or eukaryotic system prior to being used in the functional assays as described hereafter. Alternatively, prior to the construction of a coding polynucleotide from a particular mammalian cell line using an expression system, a designed tag can be added in the vector containing the coding polynucleotide that would code for a polylysine, polyarginine or other polyamino acid, or other appropriate tag. As a result, the immunogen can be purified from the expression system using the chemical nature of this tag on a particular solid support. Subsequently, these antibodies are used in function determinative assays as described hereafter.

Alternatively, the coding sequence will include in phase with the specific cloning site and the B-galactosidase a specific polynucleotide that codes for a specific peptide, such as VQGEESNDK. This peptide strongly stimulates the immune system. The insertion of a cDNA will form a fusion between the gene product coded by the B-galactosidase, the specific peptide, and the gene product coded by the cDNA. The immunogen, referred to as the fusion gene product, is comprised of a complex of the gene product coded by the cDNA, the peptide, and the B-galactosidase and is administered to a mammal of choice in the presence of an adjuvant of choice. The mixture of immunogen-adjuvant is injected as described herein in a particular tissue of a mammal.

Antibody Production Against the Gene Product of a Polynucleotide DNA Template

An injectable solution containing preferably a single or pooled set of individual polynucleotide clones from approximately 2 to 10 or more, in Tris-HCl pH7.5, 10 mM and EDTA 1 mM, are injected to immunize a mammal or mixed with a biological stimulant to boost the immune system and to provide an improved humoral response. The polynucleotide DNA for each clone is present in the injectable solution at approximately 1 micro gram per 1 microliter (1 μg/1 μl). 10 to 1000 microliters are administered to a mammal, depending on the number of combined clones and the method of administration, i.e., injected or inhalated, or in other form. Mammals are preferably non-human mammals, usually rodents and most preferably mice of two different genetic haplotypes such H₂^{d}, H₂^{b} or other genetic background. The animals are preferably 8 weeks old but could be older or younger, as long as the age does not compromise the immune response. Animals are anesthetized with ketamine/xylazine 40 mg/kg using 1 ml syringe with a 22 gauge needle. If animals are immunized with a single polynucleotide coding for a single gene product, approximately 100 microliters of DNA solution, equivalent to 100 micrograms, is injected into the mammal. Multiple injection sites are often used for each animal. The injections of DNA solution could be also carried out intramuscularly, intraperitoneally, intravenously or otherwise. The immunized mammals are cared for humanely in a clean animal facility according to the regulations in practice for the animal care and use committee and the National Institute of Health Guidelines. Four to six weeks after the first administration, the serum of the animal is harvested and the serum, or the antibody components thereof, are used in a function determinative assay, including at least one immunoassay as described herein. An assessment of the humoral reaction will determine whether the mammal should be challenged with another dose of the administered material or the serum harvested repeatedly for use in further function determinative assays. The immunizations proceed until the specific immunological reaction is judged sufficient in specificity and strength against the gene product coded by the administered polynucleotide.

The present invention will be further described with reference to the following examples which include assays for use in the basic methodology of the invention. However, it is to be understood that many additional assays are recognized by those skilled in the art and the present invention is not limited to such examples unless otherwise specified.

### EXAMPLE 1

In vivo Expression of DNA Injected Directly into the Muscles of Mice

The polynucleotide injected in the mammal is a DNA template prepared by the methodology as described above. The vector construct contains the B-galactosidase gene under the control of SV40 origin early promoter and with a polyadenylation signal. The B-galactosidase is used as a reporter gene. This vector is designated pCH110/LacZ. The quadriceps muscles of mice were injected with either 100 micrograms of pCH110/LacZ. DNA plasmid into the muscle tissue at two different sites to test for the expression of B-galactosidase enzyme activity.

Five to six week old female and male Balb/C mice were anesthetized by intraperitoneal injection with 0.3 ml of 2.5% Avertin. A 6.1 ml solution of DNA template in Tris HCl, pH8 10 mM, and EDTA 1 mM was injected in the quadricep muscle of the mice using a 1 cc syringe through a 27 gauge needle over one minute, approximately 0.5 cm from the distal insertion site of the muscle into the knee and about 0.2 cm deep. Injection sites were shaved and circled with chinese ink to localize the site of injection in the future. Injected animals were euthanized by cervical dislocation at 5, 10, 15, 30, and 40 days after the injection of pCH110/LacZ DNA template.

Muscle extracts were prepared by excising the entire quadriceps, mincing the muscle into 15 ml containing 5 ml of a lysis solution (20 mM Tris HCl, pH7.5, 2 mM MgCl₂ and 2.5% of tripsin), and grinding the muscle with a plastic pestle for five minutes in order to ensure complete disruption of the muscle cells. The muscle cells were rinsed twice with PBS (137 mM NaCl, 2.7 mM KCl, 4.3 mM Na₂HPO₄ 7H₂O, 1.4 mM KH₂ PO₄ and plated at a concentration of 5×10⁵ cells in DMEM medium containing 10% of Fetal Calf Serum in a 10 cm petri dish. Five hours later the culture medium is removed and the cells are washed twice with PBS fixed in 2% paraformaldehyde 0.2% glutaraldehyde and stained for B-galactosidase expression using a solution of: 5 mM of K₃Fe(CN)₆, K₄Fe(CN)₆, 2 mM MgCl₂ and 1 mg/ml of 4-Cl-5-Br-3 imunolyl-B-D-galactosidase in dimethyl sulfoxide.

Approximately 120 cells per 1000 cells were stained blue documenting the expression of B-galactosidase enzyme, in the injected site with DNA template. Control muscle was injected only with a PBS sucrose solution that did not show any background staining. Positive B-galactosidase staining within some individual muscle cells was at least 1.2 mm deep on serial cross-sections, which may be the result of either transfection into multiple nuclei or the ability of cytoplasmic proteins expressed from one nucleus to be distributed widely within the muscle cell. Longitudinal sectioning also revealed B-galactosidase staining within muscle cells

### EXAMPLE 2

Site of In Vivo Expression of a Reporter Gene: The B-Galactosidase Used As a Gene Template DNA Injected Directly Into the Muscles of Mice

Expression of an exogenous gene was evaluated using a DNA template bearing the B-galactosidase gene and named herein a reporter gene. In this experiment, the following parameters were analyzed and their effect on the expression of the reporter gene: (I) varying quantities of DNA template were used for immunization: 2, 5, 10, 20, 50, 100, and over 100 μg of DNA (II) the relationship between the site of injection (muscle, skin, lung, intradermal, vaginal) of DNA template and the level of expression of the reporter gene, (III) the comparison of the level of expression using the reporter gene under the control of different promoters: CMV, LTR, RSV, Actin, PGK, DHFR, SV40 early promoter, (IV) assessing the capability of the DNA template bearing polynucleotide sequences to facilitate the integration (containing LTR element), (V) the earliest time of expression of the reporter gene: 1, 2, 3, 4, 5, 6, 7, and 8 weeks, and (VI) the effect of the quality of DNA using the various known methods for the preparation of DNA template on the expression of the reporter gene.

Balb/c and C57 Black/6 mice males and females, at least 6 weeks old were shaved in the site of injection. The contemplated site is surrounded with a small circle using Chinese ink, to localize the site of injection. Animals injected with DNA templates bearing the reporter gene as described above, or with PBS sucrose solution for control animals, using 1 ml syringe and a 27 gauge needle. Immunized animals and controls were euthanized at the designated time and tissue samples were examined for the expression of B-galactosidase enzyme. Tissues were removed and cut in small pieces with a sterile blade riser, then grounded to disperse the cells. Cell suspension is carried out using PBS pH7.5. Cells were centrifuged and pelleted in a 50 ml coming tube at 2000 rpm at 4° C., for 10 minutes using top table Jouan centrifuge. Cells are plated in DMEM, 10% fetal calf serum, 100 μg of ampicillin, 100 μg streptomycin and incubated for culture in standard conditions for 6 hours. Cells were then washed with PBS and stained as described in Example 1. Cells expressing B-galactosidase enzyme are stained blue for comparison to control cells for the same tissue. Blue cells are counted in each cell type to reflect the degree of expression of the reporter gene with respect of the different parameters from (I) to (VI), outlined above.

### EXAMPLE 3

Histopathological and Immunohistochemical Examination of Lac Z Expression in Mice Immunized with DNA Template and in Control Animals

Immunized mice were euthanized by cervical dislocation and different tissues (muscle, skin, and lung) were removed and processed from both immunized animals and control littermates. Tissues were fixed in 10% phosphate-buffered formalin for a minimum of 24 hours. Tissue was processed by paraffin infiltration in sagittal orientation. Sections were cut on the rotary microtome at 6-8 microns, and routine histologic examination was done after staining with hematoxylin and eosin.

For LacZ immunohistochemical staining, paraffin sections of the different tissues described above were deparaffinized and rehydrated through xylol and graded alcohols. Sections were processed in citrate buffer (pH6.0) using a microwave oven seven times for 3 minutes. Endogenous peroxidase was quenched by treatment with 6% hydrogen peroxidase in methanol for 10 minutes. Sections were washed three times in 0.1 M PBS (pH7.5) for 5 mins. followed by a 20-minutes incubation with 10% normal horse serum in 0.1 M phosphate buffer (pH7.5). Immunohistochemical detection of B-galactosidase was performed using mouse monoclonal antibodies against B-galactosidase enzyme. Detection of primary antibodies was performed using routine diaminobenzidine-peroxidase techniques. After the reaction, the slides were washed in distilled water, counter stained with eosin, dehydrated in graded alcohols and xylol, and mounted. Sections were evaluated for the expression of B-galactosidase enzyme.

### EXAMPLE 4

Generation of Specific Antibody Against Gene Product by Immunization of Mice with Corresponding Polynucleotide Gene Sequence

Animals, males, and females used in this experiment were 8 weeks minimal and up to one-year old. Fifty animals were immunized with one 100 μg corresponding to a single cDNA clone. Other animals were immunized with a combination 2, 3, 4, 5, 6, 7, 8, 9, 10 cDNA clones, using 100 μg from each cDNA clone. In both cases, animals received a single immunization. The DNA were prepared according to the methodology described above for DNA template preparation.

Six to eight weeks later the animals subjected to single immunization were bleeded and sera analyzed by ELISA, immuno-cell-staining on fixed cells, and immunotransfer for both the specific reaction against gene product in cell extract and for the antibody titer in general.

DNA template from a cDNA expression library using regulatory elements for mammalian expression (CMV promoter) is prepared as described previously in DNA template preparation. Animals are immunized either with single DNA template corresponding to a single gene, or with a single injection of a combination of multiple DNA templates as described hereafter.

Single immunization, two animals for each clone (100 micrograms per mouse in total were injected)

1+2:2 animals

1+2+3:2 animals

1+2+3+4:2 animals

1+2+3+4+5:2 animals

1+2+3+4+5+6:2 animals

1+2+3+4+5+6+7:2 animals

1+2+3+4+5+6+7+8:2 animals

1+2+3+4+5+6+7+8+9:2 animals

1+2+3+4+5+6+7+8+9+10:2 animals

Animals were bled weekly after the immunization, starting one week after the immunization. Sera were tested as described above for the presence of specific antibodies against the gene product of the polynucleotide.

### EXAMPLE 5

Determination of Antibody Titer by ELISA in Mice Injected with DNA Template

Antibody titer was evaluated at two levels (i) for the total concentration of immunoglobulin, and (ii) for the specific antibody against the gene product in mammalian cell extract.

Coating antigen solution prepared in 100 mM carbonate/bicarbonate buffer pH 9.5, is composed of anti-mouse immunoglobulin. The final concentration of antigen is usually 0.2 to 10 μg/ml. Using a multichannel pipet 50 μl of antigen solution are added into each well of an Immulon microtiter plate. Plates are sealed and incubated at room temperature for two hours or overnight at 4° C. Then plates are washed with PBS three times, then gently flipped faced down onto several paper towels laying on the benchtop. Residual binding capacity of the plates are blocked by adding 200 μl of blocking buffer (0.05% Tween 20, 0.25% BSA in PBS) to each well and incubated for 30 min. at room temperature, then washed three times with PBS. 50 μl of the first antibody were diluted in blocking buffer (1/100, 1/200, 1/300, 1/500, 1/1000, etc.) are added to each of the coated wells and incubated at room temperature for hours. Plates are washed three times with PBS as described previously, then 50 μl per well of HRP-linked secondary antibody diluted in blocking solution are added to each well and incubated for 1-2 hours at room temperature. The OPD reagent (SIGMA) is prepared according to the manufacturer instructions. Tablets are warmed at room temperature prior to dissolution (1 tablet of OPD and 1 tablet of urea-H₂0₂) in 20 ml. of distilled water. 50 μl of this solution are added per well and plates are incubated until yellow-orange coloration develops. The evaluation of the specific reaction is examined at 450 nm using a plate reader. The antibody titer is calculated with comparison with control wells performed in parallel reactions.

### EXAMPLE 6

Determination of Antibody Titer by ELISA in Mice Injected with DNA Template Against a Gene Product in Mammalian Cell Extract

Antibodies from immunized animals with a DNA template coding for a gene product and from control animals are examined for specific antibodies against gene product in mammalian cell, using a total cell extract. Determination of antibody titer was performed by ELISA as described in Example 5. The coating antigen solution was composed of total protein extract prepared from human cell lines.

### EXAMPLE 7

Antibody Production in Mice Directly Injected with a DNA Template Coding for a Known Gene

Each mouse was injected with a total of approximately 100 μg of a plasmid construct consisting of human albumin gene, driven by a cytomegalovirus (CMV) promoter. The DNA was injected in the quadriceps muscle (50 μg/muscle)/mouse according to the methods described previously. Two mice were injected in the quadriceps muscle with the same amount of DNA template, the back bone plasmid DNA in PBS. Blood samples were obtained prior to the injection (Day 1) and then at the following times: 5, 10, 20, 30, and 40 days post injection. The serum from each sample was serially diluted and assayed in a standard ELISA technique for the detection of the specific antibody against the gene product, using human protein extract.

### EXAMPLE 8

Gene Product Localization in Fixed Cells

Desired mammalian cells are grown in 96-well plates at 60% confluency, then fixed with formaldehyde 4%. Cells are then permeablized using 200 microliters of permeablization buffer, (0.2% triton×100, 10% normal horse serum in PBS 1×) for 1 hour at room temperature (RT). The antibody to be tested is added at 1 microliter of serum/well (1/200 dilution) or lower if required herein, and incubated overnight at 4° C. Cells are then washed three times for 5 minutes each with PBS and incubated with 100 microliter/well of the biotinylated secondary antibody against mouse IgG (Vector Laboratory, vectastain kit) 1/200 dilution using the permeablization buffer containing horse serum. 96 well plates are kept for 1 hour at room temperature, then washed 3 times with PBS. Cells are then incubated in 100 microliters/well of 1/100 dilution of each of the avidin-biotin respectively in the permeablization buffer for 1 hour at room temperature and washed three times in PBS. Diaminobenzidin (DAB) solution is prepared according to manufacturer instructions (SIGMA). One tablet of DAB and one tablet of Urea/Hydrogen peroxide are dissolved in 15 ml. of distilled water, from which 50 ml are added to each of the 96 wells. The positive staining reaction is visualized under the microscope. The reaction is stopped when satisfactory staining is reached by washing out the DAB with 200 microliters of PBS. The localization of the gene product in the cell is examined for each raised single antibody against a single gene product coded by the polynucleotide sequence.

### EXAMPLE 9

Imnmunodetection of Gene Products in Cell Extracts

Mammalian cells (about 10×10⁶) were grown in culture in DMEM, fetal calf serum 10%, Streptomycin and penicillin 100 μg/ml until 80% confluency. Cells are then harvested and washed twice with PBS, resuspended in phosphate buffer (pH8.0) and disrupted in the following buffer: Tris-HCl, pH7.5, 50 mM, EDTA 2 mM, NaCl 100 mM, NP40 1%, and vanadate 1 mM, in addition to the following proteases inhibitors PMSF, Aprotinin, Leupeptin at 1, 2, and 4 mM respectively. The cell lysate was centrifuged for 5 minutes at 14,000 rpm, and protein content was determined by standard method. Total proteins (1.0 mg) were subjected to electrophoresis on 12% SDS-polyacrylamide preparative gel, and transferred onto nitrocellulose filter. After transfer, the membrane was saturated for one hour in TNE/Tween (Tris-HCl, pH7.5, 10 mM, EDTA 2.5 mM, NaCl 50 mM, Tween-20 0.1%, containing 25% dried non-fat milk). Then, the membrane was cut off in different strips of 0.5 centimeter wide each. Then, each strip was incubated for 1.5 hour with mouse antibody raised against either single polynucleotide DNA template or a mixture of several clones, at 1/500 dilution in the blocking buffer. The blot was washed five times in the same buffer described above and incubated with sheep anti-mouse IgG antibodies conjugated with horseradish peroxydase, diluted 1/1000 in the blocking solution for 1 hour. Then, strips were washed 5 times 10 minutes each in TNE/Tween without milk. Anti-antibodies were visualized using the alkaline phosphatase conjugated substrate solution from Pierce named SuperSignal™ CL-HRP Substrate System. Strips are placed between two transparent plastic sheets and exposed to X-Omat AR film for several seconds or a few minutes until satisfactory detections of the specifically recognized gene products are achieved.

Function Determinative Assays

Antibodies generated by the practice of this invention are used in a function determinative assays, which is comprised of at least one immunoassay using antibodies (GPTs) to the gene product to determine the function of the gene product in a selected biological process or physiological condition. The antibodies thus tested in the assays selected from the following examples or others, identify or discover gene sequences and gene products in normal and pathological conditions. The practice of this invention simultaneously correlates the coded gene product, coding gene sequence, the antibody to the gene product (GPT), the cellular localization, the cell type, an assessment of the molecular weight, and the function in normal and pathological conditions.

A combination of assays can be used to analyze a wide variety of parameters in different cell types such as transformed and nontransformed cell lines of different differentiated or undifferentiated cell types (tumor cells, neuronal cells, muscular cells, liver, skin, colon, intestine, testis, ovary, heart, muscle, pancreas, spleen, colon, bladder, brain, lung, prostate, endothelial cells, kidney, synovial membrane, bone marrow, stem cells, pancreas, blood cells (hematopoeitic cells), lymphoid cells, adrenal gland, adipose cells, candreocytes, testis, parathyroid, salivary gland, breast, ovary, placenta, uterus, gall bladder, and other mammalian cell types). Additional, cancerous or tumor cell lines for each may be analyzed. Normal cells may also be subjected to any of a variety of changes in condition related to a pathological condition such as inflammation, autoimmunity, infection by viral, bacterial, fungal, parasitic or other organism, degeneration, apoptosis, uncontrolled growth or mutation, etc. The function determinative assays that can be applied to tumor cells can also be applied to stem cells, neuronal cells, endothelial cells, hepatic cells, candriocytes and any other type of cells in a mammal. The application of these assays to normal and pathological cell types will allow the discovery of crucial molecules or compounds that can be either modulated or targeted to control a pathological condition. The function determinative assays can be applied to any biological process, pathology, or physiological condition to reveal strategies for the discovery or manufacture of agents for diagnosis and therapeutic intervention.

The following is an exemplary group of assays and strategies that may be used in the basic methodology of the invention. The following examples and others not expressly set forth but known to those in the art, can be readily applied to any selected biological process, physiological condition, typically a pathological condition indicative of a disease in a human.

Assays on Tumor Cells

Growth and Inhibition

Growth and inhibition assays may be performed on different tumor cell lines derived from any tissue or organ as mentioned herein, and particularly in cancers with a high incidence nd mortality in humans such as breast, lung, liver, prostate, kidney, brain, colon, bladder, osteosarcoma, colon, ovary, spleen, etc. Generally, tumor cells and normal cells of the same tissue type are used as controls and are grown in culture in 96 well plates at an exponential phase for a minimum of 24 hours, or more if needed. Antibodies contained in the sera of the immunized mammal are added to both normal and tumor cell samples at the same concentration and kept in culture for 48 hours in the presence of ³H Thymidine. Tumor and control cells are washed and assessed for their growth status by direct counting of ³H thymidine incorporation.

The binding of the antibody to a gene product that controls the pathology in the assay will demonstrate that the physiological phenomenon, such as growth inhibition, cell death, or others is directly related to a gene sequence encoding the gene product against which the antibodies were made. Therefore, this approach identifies the gene sequences and gene products involved in growth inhibition and cell death in these cells, and establishes a one-to-one correlation between gene product, gene sequence, and gene function.

If the antibody binds to a critical hormone, necessary for growth of cancer cells and blocks the function of the hormone, a pathway for therapeutic intervention has been identified to control the growth of the tumor, as well as the means by which the therapeutic intervention is delivered, e.g., the antibody.

Specific Markers on Tumor Cells

Tumor cells possess a variety of characteristics including the expression, the absence, the up-regulation, and the down-regulation of different components as compared to normal cells. Indirect immunofluorescence using specific antibodies on tumor and control cells allows the identification of specific markers for tumor cells that control important biological functions. The identification of such markers can be used for diagnosis, immunotherapies and drug discovery to specifically target these cells.

One important embodiment of the present invention discovers the localization of the gene product encoded by a polynucleotide that is highly expressed in tumor cell lines, but not highly expressed in normal cells. Such information is crucially important to design, develop, and apply additional assays to design a suitable therapeutic intervention which may utilize an extracellular, intracellular, or excreted gene product.

Extra and Intracellular and Secreted Gene Products

A strategy enabled by the present invention identifies novel genes that code for important gene products by virtue of the action of GPT in vitro that will result in the physiological changes of the cell. In mammals, the gene products could be either secreted components outside the cell such as growth factors, hormones, neuropeptides and ligands cell surface components such as proteins, glycoproteins, phospholipids, glycolipids and receptors, and intracellular components that may be found in sub-cellular compartments: nucleus, golgi, endoplasmic reticulum, etc. These gene products, regardless of their cellular localization, can be identified by specific antibodies and those that are accessible in living cells can eventually be blocked in their physiological functions by antibody intervention. Furthermore, gene products inside the cells are generally regulated through cascades of molecular events that transit through membrane cellular components. Consequently, the binding of one antibody to a such cellular component involved in cascade molecular events, where the real effect takes place into the nucleus, may block this effect into the nucleus while it is acting at a distance at the level of the cell surface.

A variety of methods such as immunofluorescence (IF), FACS scan (FACS), and flow cytometry (FC) will be performed on mammalian cells in suspension and/or adherent cell lines, as described in current protocols of Immunology, Wiley Interscience, by John E. Colligan et al. The source of these cell lines are derived from any tissue and from any mammals.

The function determinative assays described herein identify specific components (compounds, markers, factors, effectors, etc.) associated with the following: biological processes and physiological conditions, among others:

1. specific components in tumor cell lines (prostate, breast, lung, bladder, colon, liver, ovary, teratocarcinomas, lymphomas, and other tumor types),

2. specific components in undifferentiated cell lines of different cell types,

3. specific components in differentiated cell lines of different types,

4. specific components in particular tissues,

5. specific components related to pathological conditions,

6. specific components related to infectious conditions (viral, bacterial, fungal, Parasitic and others),

7. specific components related to inflammatory conditions,

8. specific gene products related to autoimmune diseases, and

9. specific gene products related to neurodegenerative disorders.

It should be understood by those skilled in the art that the present invention can be applied to each of the above examples, among others. The practice of the basic methodology of the invention, applied to the item 1 above, e.g., tumor cells, demonstrates the application of the basic methodology to any biological process or physiological condition, and will result in the following data according to the process or condition.

a. the identification of gene products either secreted, on the cell surface, or in the intracellular space, that are specifically expressed in tumor cell lines compared to normal cell lines,

b. the identification of gene products either secreted, on the cell surface, or in the intracellular space, that are specifically absent in tumor cell lines compared to normal cell lines,

c. the identification of gene products either secreted, on the cell surface or in the intracellular space that are specifically up-regulated in tumor cell lines compared to normal cell lines,

d. the identification of gene products either secreted, on the cell surface or in the intracellular space, that are specifically down-regulated in tumor cell lines compared to normal cell lines,

e. the identification of gene products that induce the growth of tumor cells ,

f. the identification of gene products that inhibit the growth of tumor cells,

g. the identification of gene products that induce the death of tumor cells, and

h. the inhibition of the invasiveness of tumor cell in agar.

The gene sequences corresponding to the targeted gene product, the gene product, and the antibody in the above steps, (a) through (h) are each subject to use for therapeutic intervention, including drug discovery, molecular therapy, cellular therapy, or diagnosis.

The characteristics cited above can be readily applicable to primary stem cells. Again, the practice of the present invention based on the function determinative assays, will lead to the following data, among others.

1. the identification of antibodies having the capacity to promote the growth or inhibition of specific cells,

2. the identification of antibodies having the capacity to promote the differentiation of these cells,

3. the identification of antibodies having the capacity to promote the change in the metabolism of these cells as measured by the change in the concentration of the metabolic enzymes,

4. a determination of changes on ion concentration such as Ca++, H+, Na+ and K+ that are indicative of important pathologies, biological process, and physiological conditions,

5. a determination of the effect on superoxide production by these cells,

6. the identification of antibodies having the capacity to intervene by drug action,

7. the identification of antibodies having the capacity to stimulate enhancement or interruption of pharmacological drug effect,

8. the identification of antibodies having the capacity to effect the production of chimokine, lymphokines and growth factor by cells in a mammal. The latter will be used on other cell types such as erythroid and lymphoid cell lineage's to test the potential of growth and/or differentiation for a variety of cell lineages, gene products, their corresponding polynucleotides, and antibodies are then readily applicable for therapeutic intervention. As noted throughout, the identification of the antibody (GPT) having the above capacities or others provides a one-to-one relationship between the function, the antibody, the gene product, and the corresponding gene sequence.

Immunological Functions

The present invention can also be applied to modulate effector immune cells and their components in the immune system. The gene product specific antibodies (GPTs) are used in function determinative assays, including at least one immunoassay, to test the effect on the effector cells in the immune system, such as dendritic cells, monocytes, macrophages, T and B-cells and their sub populations, and to discover important genes, gene sequences, and gene products that control and regulate the immune system.

1. function determinative assays test for activation, growth, differentiation, lymphokine production and toxicity (activation of serine esterase to monitor CTL activity).

2. function determinative assays test for antibody dependent cell mediated cytotoxicity.

3. function determinative assays test for stimulation of macrophages for histamine and serotonine secretion.

4. function determinative assays test for stimulation of macrophages for superoxide formation.

5. function determinative assays test for abolition of histamine and serotonine secretion, and superoxide formation by macrophages and monocytes.

6. function determinative assays test for the effect on degranulation of mast cells.

7. function determinative assays test for effect on control of the inflammatory process induced in an experimental model.

8. function determinative assays test for enhancement of microbicidal effect.

9. function determinative assays test for measurement of intracellular ions by flow cytometry.

10. function determinative assays test for the effect on the up- or down-regulation of adhesion molecules.

Vascular Endothelial Cell

Vascular endothelial cells derived from umbilical vein from umbilical cord of new born are tested for their sensitivity to GPTs, both for intracellular ions and for their growth and their capacity to up- and down-regulate the concentration of adhesion molecules.

Cationic and Anionic Exchange

GPTs are tested to identify ions channels in neuronal and muscular cells and to test their effect on their modulation.

1. GPTs are tested to block ion channels in these cells.

2. GPTs are tested to assess the potential effect on neurogenesis and chemotactism.

3. GPTs are tested to prevent neurodegeneration.

4. GPTs are tested to promote neuronal differentiation.

Pathogen Infection

Infection of mammalian by pathogens occurs through interaction between pathogen ligands (virus, bacteria, fungus, parasite) and cell receptors.

1. GPTs are tested to identify and block receptors on the cell surface to prevent the interaction between pathogen and cellular receptors.

2. GPTs are tested for their potential to enhance phagocytosis of pathogens and infected cells and to increase cellular toxicity of the immune system by acting on a gene product molecule on the cell surface.

The above examples related to tumor cells, stem cells, effector immune cells, vascular endothelial cells, and infectious pathogens are merely illustrative examples among many others within the scope of the invention based on the practice of the basic methodology employing the following function determinative assays and others known to those of ordinary skill in the art. The following examples are also capable of identifying novel gene sequences, gene products, and their corresponding antibodies. The identification of these biological components enable the creation of therapeutic and diagnostic agents and strategies the application of the following examples, while not so limited, are within the scope of the present invention.

### EXAMPLE 10

Enzyme-Linked Immunosorbent Assays

The present invention includes different ELISA systems for the detection of specific antibodies, soluble antigens, or cell-surface antigens. In all systems, soluble reactants are removed from solution after specifically binding to solid-phase reactants. Solid-phase reactants are prepared by adsorbing an antigen or cell-associated molecules or antibody onto plastic microtiter plates. In all protocols, the solid-phase reagents are incubated with secondary or tertiary reactants covalently coupled to an enzyme. Unbound conjugates are washed out and a chromogenic or fluorogenic substrate is added. As the substrate is hydrolyzed by the bound enzyme conjugate, a colored or fluorescent product is generated. Finally, the product is detected visually or with a microtiter plate reader as described in the Current Protocol of Immunology; Edited By John E. Coligan et al., (CPI), Wiley interscience.

### EXAMPLE 11

Direct Cellular ELISA to Detect Cell-surface Antigens

The expression of cell-surface antigens or receptors is measured using existing antibodies or other ligands specific for cell-surface molecules. Cells are incubated with enzyme conjugated to antibodies that are specific for a cell-surface molecule. Unbound conjugate is washed away and substrate is added. The level of antigen expression is proportional to the amount of substrate hydrolysis. This procedure can be as sensitive as flow cytometry analysis in quantitating the level of antigen expression on a population of cells (UNITS 5.1-5.4)/CPI.

### EXAMPLE 12

Indirect Cellular ELISA to Detect Antibodies Specific for cell-surface Antigens

An indirect cellular ELISA assay is designed to screen for antibodies specific for cell-surface antigens. Antibodies against surface antigens are detected by incubating whole cells with a test solution containing the primary antibody. The unbound antibody is washed away and the cells are then incubated with an enzyme conjugated to antibodies specific for the primary antibody. Unbound enzyme conjugate is washed away and substrate solution added.

### EXAMPLE 13

In vitro Assays for Mammalian Cell Function

The present invention includes methods for assessment of beta cell activation (i.e., induction of a rise in intracellular calcium, an increase in cell size, and enhanced expression of MHC class beta antigens) as presented in UNIT 3.9./CPI.

Procedures for measuring cell proliferation induced by antigens, mitogens, anti-TCR and anti-Thy-1 antibodies, and direct triggering of intracellular activation pathways with phorbol ester and calcium ionophore are used as described in UNIT 3.12./CPI of CPI. The protocols used for the induction of proliferation also form the basis for the establishment of cultures that can be used to measure secretion of cytokines with methods presented in Chapter 6, of the Current Protocol of Immunology. Limiting dilution techniques were designed to overcome this problem and can be used for measurements of the frequency of both lymphokine-producing and cytotoxic T cells, as described in UNIT 3.15./CPI

### EXAMPLE 14

Flow Cytometry

The present invention includes the use of flow cytometry methods for analyzing the expression of cell surface and intracellular molecules (on a per cell basis), characterizing and defining different cell types in heterogeneous populations, assessing the purity of isolated subpopulations, and analyzing cell size and volume. This technique is predominantly used to measure fluorescence intensity produced by fluorescent-labeled antibodies or ligands that bind to specific cell-associated molecules. The method related to a procedure for direct and indirect staining of single-cell suspensions of lymphoid tissue or peripheral blood lymphocytes (PBL) to detect cell surface membrane antigens. Methods for flow cytometric analysis of intracellular antigens in single-cell suspensions (e.g., mononuclear cells from human or murine peripheral blood or bone marrow, lymphoid cell suspensions, cells grown in suspension cultures, or dissociated tissues) are also included.

### EXAMPLE 15

Immunofluorescence Staining of Single-cell Suspensions for Detection of Surface Antigens

Immunofluorescence staining for flow cytometric analysis involves making a single-cell suspension from lymphoid tissues or peripheral blood, successive binding steps in which cells are incubated in tubes or microtiter plates with unlabeled or fluorescent-labeled antibodies or ligands, and wash steps using excess buffer to remove unbound antibodies.

### EXAMPLE 16

Immunofluorescence Staining of Fixed and Permeablized Single-cell Suspensions for Detection of Intracellular Antigens

This method of preparing cells for intracellular staining retains the scatter characteristics of cells on the flow cytometer, preserves surface immunofluorescence, and allows accurate measurement of cellular DNA content by yielding good coefficients of variation on DNA histograms. It is therefore particularly suited for multiparameter flow cytometric analysis. For simultaneous analysis of cell-surface and internal antigens, cells that have been stained for expression of cell-surface antigens can also be stained for expression of intracellular antigens.

### EXAMPLE 17

Immunofluorescence Staining of Unfixed Cells for Detection of Intracellular Antigens

This method for preparing cells for intracellular staining, preserves immunofluorescence, and can also be used for measuring DNA content. For simultaneous analysis of surface and internal antigens, cells that have been stained for expression of cell-surface antigens can also be stained for expression of intracellular antigens using this protocol.

### EXAMPLE 18

Measurement of Intracellular Ions by Flow Cytometry

The flow cytometer can be used to measure various functional parameters that are critical to cell function in normal and pathological conditions, and are characterized by changes in the concentrations of various intracellular free ions in single living cells. Among these ions are calcium, magnesium, sodium, potassium, and hydrogen (pH). This method allows ion concentration to be correlated with other parameters such as immunophenotype and cell cycle.

### EXAMPLE 19

Use of INDO-1 AM and Flow Cytometry to Measure Cellular Calcium Concentration

In this method, intracellular ionized calcium concentration ([Ca²⁺]ᵢ) is measured using Indo-1 AM dye and ratiometric analysis. Most commercially available flow cytometers can be used to perform this assay.

### EXAMPLE 20

Measurement of Intercellular Conjugates by Flow Cytometry

The present invention also includes the analysis of fluorescently labeled cellular aggregates that can result from the incubation of the raised antibodies against the polynucleotide and a particular cell type. Conjugates can be measured by a dual-laser instrument when the extent of labeling of two cell types with fluorophores are very different. After the incubation with specific antibodies, one cell type is stained with a green-emitting fluorophore and the other with a red-emitting fluorophore. The cells are then mixed to allow conjugate formation, followed by analysis with a dual-fluorescence flow cytometer.

### EXAMPLE 21

Use of Flow Cytometry for DNA Analysis

Flow cytometry can be used to assess the cell cycle status of mammalian cells. For example, flow cytometry measures mammalian cell activation and proliferation with propidium iodide (PI) staining, and can also be combined with standard surface-marker staining for analyzing two or more subpopulations.

### EXAMPLE 22

Immunohistochemical Staining of Cultured Cells

Immunohistochemistry (IHC) can be used to evaluate a variety of immunologic reagents, including those that recognize cytokines and activation antigens as well as adhesion molecules. This procedure identifies the subcellular localization of particular antigens (e.g., nuclear, Golgi, or surface-associated antigens) in single-cell preparations. In addition, IHC of cell preparations, as with tissue sections, can be combined with in situ hybridization, in situ apoptosis, or double immunostaining to examine several parameters simultaneously. Cultured cells can also be used as a positive control for determining the optimal concentration of antibody for a transiently expressed antigen such as adhesion molecules (e.g., E-selectin). For the study of transient antigens, cells in culture can be activated and fixed at the appropriate time for optimal antigen expression.

### EXAMPLE 23

Ouantitation of DNA Synthesis Using the MTT Calorimetric Assay

This procedure employs the tetrazolium salt 3-(4,5-dimethylthiazol-2-yl)-2,5-diphenyltetrazolium bromide (MTT), which is cleaved by active mitochondria to form a formazan product that can be completely solubilized in acidic isopropanol and detected by a microtiter plate reader (Mosmann. 1983). The assay allows the detection of living and growing cells

### EXAMPLE 24

Measurement of Interleukin 3 and Other Hematopoietic Cytokines, such as GM-CSF. G-CSF, M-CSF. Erythropoietin, Steel Factor, and Flt-3 Ligand

The present invention includes the detection and quantitation of hematopoietic cytokines in biological samples and culture supernatants. Such measurements have played an important role in understanding their function and pleiotropic properties during the process of hematopoiesis. The procedure utilizes several methods for detecting and quantitating cytokines—such as the colony-stimulating factors granulocyte/macrophage colony-stimulating factor (GM-CSF), granulocyte colony-stimulating factor (G-CSF), macrophage colony-stimulating factor (MCSF, also called CSF-1), interleukin 3 (IL-3), and erythropoietin (Epo), as well as two potent co-stimulating cytokines steel factor (SLF) and Flt3/Flk-2 ligand (Flt3-L)—are described in the Current Protocol of Immunology, Wiley in the chapter 6, related to lymphokines.

The methods include cytokine bioassays based on the growth of a variety of hematopoietic progenitor cells as colonies in a semisolid culture medium and based on the enumeration of colonies (colony-forming units, CFUs) derived from granulocyte/macrophage progenitor cells (CFU-GM). These methods also include a cytokine activity assay based on the differentiation of either an erythroid progenitor (burst-forming units-erythroid, BFU-E) or a multi-potential progenitor (colony-forming unit granulocyte/erythroid/macrophage/megakaryocyte, CFU-GEMM, or CFU-Mix). These methods use normal mammalian cell populations from bone marrow, peripheral blood, cord blood, or spleen as primary targets.

### EXAMPLE 25

Detection of Hematopoietic Cytokines Using Granulocyte/macrophage Cell Growth in Semisolid Medium

This assay exploits the capacity of hematopoietic progenitor cells present in bone marrow, spleen, peripheral blood, and umbilical cord blood cells, and stem cells, to proliferate and differentiate in semisolid culture medium in response to single or multiple cytokines. Interaction between specific antibodies raised after polynucleotide injection and these cells could trigger the production and secretion of particular cytokines, which in turn will result in the multiplication and/or differentiation of particular mammalian progenitor cells to differentiate into multicellular colonies with specific morphologies. These colonies are visible using an inverted microscope and can be enumerated.

### EXAMPLE 26

Detection of Hematopoietic Cytokines Using Cytokine-dependant Cell Lines

Cell lines that are dependent on one or more cytokine for proliferation can be used to provide target cell populations. For example, FDC-P2, an IL-3-dependent cell line that can be employed to detect IL-3, will proliferate in a concentration-dependent manner. Proliferation can be measured by incorporation of [³H]thymidine (see UNIT 3.17)/CPI, reduction of MTT (see UNIT 6.3)/CPI, or colony formation in semisolid medium.

### EXAMPLE 27

Antibody Neutralizing Assay

The present invention can also use antibodies to identify molecules, such as interferon species (IFN) that are responsible for antiviral activity in a test sample, or to test the potency of a particular antibody preparation against a particular species of IFN. When antibody against a specific species of IFN binds to IFN, it neutralizes its activity. Thus, antibody-mediated reversal of IFN-induced antiviral activity is the basis for further characterization of an IFN preparation. Neutralizing activity is measured by the procedure of Havell and Spitalny (1983) as modified by Vogel et al. (1986)

### EXAMPLE 28

Chemotaxis Assay of Neutrophil Response to Chemokines

Chemotaxis may be evaluated using either a multiwell chemotaxis chamber or Boyden Chambers, which are individual chambers each containing a single chemotaxis well. The multiwell chamber used in this procedure allows a number of chemoattractants (or dilutions of the same chemoattractant) to be investigated at once.

### EXAMPLE 29

Elispot Assay to Detect Cytokine-secreting Mammalian Cells

In this procedure, the filter immunoplaque assay, otherwise called the enzyme-linked immunospot assay (ELISPOT), which was initially developed to detect and quantitate individual antibody-secreting beta cells (UNIT 7.14)/CPI, will be used. These assays will rely on the concentration of a given protein (such as a cytokine) in the environment immediately surrounding the protein-secreting cell. These cell products are captured and detected using specific antibodies, and can be quantitated visually, microscopically, or electronically.

### EXAMPLE 30

Measurement of Proliferative Responses of Cultured Mammalian Cells

This method includes the measurement of proliferative responses of mammalian cells which results form various stimuli, such as the mitogen phytohemagglutinin (PHA), phorbol esters, Ionomycin, specific antigens and others to assess their biological responses. Cell proliferation is determined by estimating incorporation of [³H]thymidine into DNA, a process which is closely related to underlying changes in cell number. This procedure includes different prototype protocols to measure the proliferation response of mammalian cells following exposure to mitogens, antigens, allogeneic or autologous cells, or soluble factors. The presence of specific antibodies in the culture of mammalian cells may enhance or block the proliferative activity of the mitogenic factors.

### EXAMPLE 31

Measurement of Polyclonal and Antigen-Specific Cytotoxic T Cell Function

This technique measures in vitro cytotoxic function of human T cells, for example, which can be accomplished by polyclonal stimulation of T cell effectors using anti-CD3 antibody, which stimulates all cytolytic effector cells, or with a specific stimulating antigen. Accordingly, two sets of assays of cytolytic T cell function are assessed by measuring anti-CD3-mediated cytotoxicity and by measuring antigen-specific cytotoxicity.

### EXAMPLE 32

Measurement of Cytotoxic Activity of NK/LAK Cells

In this assay, the capacity of human natural killer (NK) or lymphokine-activated killer (LAK) cells to lyse tumor cell targets is tested. The cytotoxicity of these effector cells is evaluated in a short-term (4-hr) ⁵¹Cr-release assay using NK-sensitive tumor cells as the targets for NK cells or NK-resistant tumor cells as the targets for LAK cells. In the basic protocol, a generic ⁵¹Cr-release assay is used in which PBMC separated on Ficoll-Hypaque gradients, purified NK cells prepared as described in UNIT 7.7,/CPI or interleukin 2 (IL-2)-activated lymphocyte populations (LAK cells) may be utilized as effector cells. Nonadherent tumor cell lines maintained in culture serve as the ⁵¹Cr-labeled target cells, but cells obtained from malignant effusions, trypsinized tumor cells from adherent monolayer culture, or freshly isolated tumor cells from surgical specimens may also be used. The procedure for labeling target cells from any of these sources with ⁵¹Cr is described. Alternatively, nonradioactive methods for labeling target cells and detecting target-cell lysis, using calcine in lieu of ⁵¹Cr, can be used.

### EXAMPLE 33

Measurement of Phagocytic Capacity of Neutrophils and Microbicidal Assay Using Lysostaphin as Described in CPI

In this procedure, purified neutrophils are mixed with heat-killed, radiolabeled Staphylococcus aureas organisms under conditions in which phagocytosis can proceed. The neutrophils and phagocytized bacteria are then digested and counted and the phagocyte-associated radioactivity is taken as a measure of phagocytosis.

This protocol combines viable staphylococci with human neutrophils in the presence of serum for varied time intervals. Bacteria that are not taken up (extracellular bacteria) are destroyed by lysostaphin and neutrophils are disrupted by hypotonic lysis, releasing ingested bacteria. Viable bacteria are then cultured on plates and the colonies enumerated.

In some situations (i.e., with bacteria other than staphylococci), it may be necessary or desirable to perform the microbicidal assay without the use of lysostaphin. In this case, the assay conditions are similar to those described in the previous basic protocol except that free bacteria and those which are associated with phagocytic cells are separated by differential centrifugation.

### EXAMPLE 34

Nitroblue Tetrazolium Test

This method is used for assessing the ability of activated neutrophils to generate reactive products of oxygen. It provides a determination of NADPH oxidase activity. Neutrophils in a drop of freshly obtained blood are allowed to adhere to a stimulus-coated coverslip and then incubated with nitroblue tetrazolium (NBT). Products of the respiratory burst, primarily superoxide, reduce the NBT to formazan, which forms a blue-black precipitate in the positive cells. Individual cells are scored microscopically for positive N-BT reactions. Binding of specific antibodies to neutrophil components may result in an increase or decrease a such activity and allow the identification of important molecule.

### EXAMPLE 35

Superoxide Formation or Inhibition: Kinetic and Static Assays

This method describes the means for obtaining precise quantitative measures on the formation of superoxide by intact stimulated neutrophils, for example, or other mammalian cells. Targeted cells and cytochrome c, or other reductants, are mixed and allowed to equilibrate. A stimulus is added and superoxide production is assayed spectrophotometrically as a function of ferricytochrome c reduction. An alternative method of the SOD-inhibitable cytochrome c reduction assay involves the measurement of the total amount of superoxide formed after a fixed time. This approach permits the measurement of superoxide formation in multiple specimens, in which the assessment of kinetics would be unwieldy.

### EXAMPLE 36

Superoxide and Hydrogen Peroxide Formation or Inhibition: Subcellular Fractions

These methods measure oxidase reactions in a cell-free systems using subcellular fractions. Mammalian cells are mixed with the protease inhibitor diisopropylfluorophosphate (DFP), or with other appropriate protease inhibitors, after which they are disrupted by nitrogen cavitation. Intracellular organelles are recovered, and are incubated with an activating agent, followed by the addition of NADPH to initiate the reaction. Reduction of cytochrome c is recorded spectrophotometrically. As for the kinetic assay, the SOD-inhibitable reduction of ferricytochrome c is used as a measure of superoxide formation to assess the mechanism of action in enzyme activation or catalysis.

Alternatively, similar method are used to measure the hydrogen peroxide formation by stimulated mammalian cells. The assay determines the rate of increase in fluorescence by the horseradish peroxidase (HRPO)-catalyzed peroxidation of homovanillic acid (HVA) to a fluorescent dimer (2,2′-dihydroxy-3,3′-dimethoxydiphenyl-5,5′-diacetic acid).

### EXAMPLE 37

Measuring Degranulation of Mast Cells

The present invention includes methods for measuring exocytosis of preformed mediators from secretory granules as an indication of IgE receptor-mediated activation of mast cells. The first method corresponds to the measurement of biogenic amines (serotonin and histamine) or for either one of them serotonin or histamine, secreted by activated mammalian mast cells. Another alternate methods is used for measuring the release of b-glucuronidase, which is released during degranulation. Methods for assaying other enzymes released during degranulation, such as b-hexosaminidase and tryptase, are also included.

### EXAMPLE 38

Antibody-dependent Cell-mediated Cytotoxicity (ADCC)

Antibody-dependent cell-mediated cytotoxicity (ADCC) is used as a test for immunocompetence in effector cells or to test the activity of an antibody to mediate ADCC, based on effector cells with receptors for the Fc portion of immunoglobulin. The basic protocol is a method of measuring ADCC effector activity in lymphoid cells (peripheral blood mononuclear cells, or PBMC) that employs ⁵¹Cr-labeled target cells. The three components are mixed in microtiter-plate wells and lysis of the target cells is detected by measuring the release of radioactivity into the cell supernatant. Procedures for preparing anti-target cell antiserum and ⁵¹Cr-labeled target cells are also used. These methods can also be readily applied to the measurement of other types of effector-cell populations, such as whole lymph node/spleen cell population or purified killer cell populations from various sources.

### EXAMPLE 39

Measuring Cell Adhesion to Immobilized Ligands in the Presence of Stimulatory or Inhibitory Antibodies

This assay is designed to test the effect of antibodies on the adhesion of mammalian cells in suspension to purified ligands or cell monolayers. These antibodies can either inhibit or enhance the adhesion being measured.

This method measures adhesion, for example, of mammalian T cells to purified mammalian integrin ligands, such as the extracellular matrix protein fibronectin or biochemically purified ICAM-1 (intercellular adhesion molecule 1). Alternatively, any combination of cells and adhesive ligands listed in Table 7.28.1, in page 7.28.1 in the Current Protocol of Immunology, Chapter 7: Edited By John E. Coligan et al., can be used. The static adhesion assay involves five steps: (1) immobilizing the target ligands in microtiter plate wells, (2) adding ⁵¹Cr-labeled lymphoid cells to the wells containing the immobilized ligand at 4C in the presence or absence of activating agents, (3) initiating adhesion by raising the temperature to 37° C., (4) washing away nonadherent cells, and (5) determining the percentage of adherent cells by lysing them and measuring ⁵¹Cr gamma emissions.

In the present invention the method includes the measurement of modulation of functional activity of many adhesion molecules that can be modulated by specific antibodies to ligands and adhesion molecules. For example, stimulating human T cells with phorbol ester rapidly up-regulates the functional activity of integrin receptors. Examples of agents that can be used to modify the adhesiveness of various cell types are listed in Table 7.28.3., in page 7.28.1 in the Current Protocol of Immunology, Chapter 7: Edited By John E. Coligan et al. The agents that will be used in this method are not limited to those in this table 7.28.3.

This assay will be used with other types of cells, activation stimuli, or adhesive ligands.

### EXAMPLE 40

Analyzing Cell Adhesion to Adherent Cell Monolayers

This assay is used to measure the binding of lymphocytes to adherent cell monolayers such as cultured human umbilical vein endothelial cells (HUVEC), fibroblasts, or Chinese Hamster Ovary (CHO) cell transfectants, using a 96-well microtiter plates. This assay can be used to assess the antibody-blocking capacity of various adhesion molecules to a specific cell-cell interaction. For example, activated HUVECs express E-selectin, VCAM-1 (vascular cell adhesion molecule), and ICAM-1 (intercellular adhesion molecule 1), and each of these molecules participates in the adhesion of human T cells to activated HUVECs. The specific contribution of each adhesion molecule to the overall adhesion observed is determined by conducting antibody inhibition experiments. Typically in such complex interactions, antibodies that block a single adhesion receptor-counterreceptor system—such as LFA-1 (leukocyte function antigen 1)/ICAM-1—only partially inhibit the total adhesion observed. The use of various antibody combinations can thus be useful in elucidating the contribution of multiple adhesion receptors to a single cell-cell interaction.

### EXAMPLE 41

Measurement of the Effects of Substrate-specific Antibodies or Activating/inhibiting Substances on Cell Adhesion

The present invention includes the use of antibodies against molecules on the substrate and/or agents that activate or inhibit activation of the substrate, including pharmacological drugs and cytokines. For example, histamine can be administered to up-regulate the surface expression of P-selectin on endothelial cells (Jones et al., 1993). Antibodies against P-selectin may then be used to identify the role of P-selectin in adhesion to endothelial cells (Jones et al., 1993). This method will be used to identify potential drugs that act on cell adhesion molecules.

Antibodies, as well as activating or inhibiting agents, may be used with perfusing cells (i.e., cells in suspension). For example, antibodies against L-selectin, LFA-1, and Mac-1 may be used to identify the roles of selectins and integrins in the adhesion of neutrophils (Smith et al., 1991).

### EXAMPLE 42

Immunoblotting and Immunodetection

Biochemical methods that test the gene product recognized by the specific antibodies are also used. For example, immunoblotting (often referred to as western blotting) is used to identify specific antigens recognized by antibodies. Protein samples are solubilized, usually with sodium dodecyl sulfate (SDS) and reducing agents such as dithiothreitol (DTT) or 2-mercaptoethanol (2-ME). Following solubilization, the material is separated by SDS-PAGE (UNITS 8.4 & 8.5 in the Current Protocol in Immunology, John E. Coligan et al., J. Wiley and Sons Inc.). The antigens are then electrophoretically transferred in a tank or a semidry unit to a nitrocellulose, PVDF, or nylon membrane, or to any other support membrane.

The transferred proteins are bound to the surface of the membrane, providing access for reaction with immunodetection reagents. All remaining binding sites are blocked by immersing the membrane in a solution containing either a protein or detergent blocking agent. After probing with the primary antibody, the membrane is washed and the antibody-antigen complexes are identified with horseradish peroxidase (HRPO) or alkaline phosphatase enzymes coupled to the secondary anti-IgG antibody (e.g., goat anti-rabbit IgG). The enzymes are attached directly or via an avidin-biotin bridge to the secondary antibody. Chromogenic or luminescent substrates are then used to visualize the activity.

### EXAMPLE 43

Biotinylation and Analysis of Membrane-Bound and Soluble Gene Products

Methods to couple a biotin moiety to gene products and their detection using a variety of commercially available avidin or streptavidin conjugates are also used. These methods include the biotinylation of cell-surface proteins and proteins in solution.

Cell-surface proteins are labeled by a short incubation with an aqueous solution of sulfo-NHS-biotin. Unreacted biotin is quenched by incubation with serum-free culture medium, then cell membranes are solubilized with detergents and the lysates subjected to SDS-PAGE. The gel-resolved labeled proteins are transferred to nitrocellulose membranes by protein blotting. Biotinylated proteins are then detected by reaction with streptavidin-conjugated alkaline phosphatase or horseradish peroxidase followed by visualization with an appropriate substrate.

Alternatively, biotin labeling of gene products in solution, such as those secreted in culture medium by a particular cell type for example, can be useful to indicate the biochemical status of the said particular cell line. In this method, the gene product in solution is labeled by incubation with sulfo-NHS-biotin. Free sulfo-NHS-biotin is then rapidly removed by size-exclusion chromatography.

### EXAMPLE 43

Analysis of Inositol Phospholipid Turnover and Phosphorylation During Mammalian Cell Activation and Differentiation

The present invention involves the measurement of phospholipase activation to study the signaling mechanism of cells in normal and pathological conditions. The interaction of specific antibodies and a gene product on the cell-surface of a particular cell line may result in various changes in that cell line. For example, a such interaction can involve the activation of a phospholipase. Receptor-mediated activation of phospholipase C (PLC) leads to the hydrolysis of membrane inositol phospholipids, generating diacylglycerol (DAG) and water-soluble inositol phosphates.

Protein phosphorylation is another method that can be used to assess intracellular signaling. The methods used herein refer to those described in the Current Protocol in Immunology, UNITS 11.2 & 11.3. By metabolically labeling cells with ³²P, the phosphorylation state of cellular proteins can be analyzed and changes therein occurring with receptor stimulation can be examined (UNIT 11.2. in the Current Protocol in Immunology, John E. Coligan et al.). Phosphoamino acid analysis can be used to determine whether serine, threonine, or tyrosine residues are phosphorylated, thereby providing information about the kinase involved. For example, analysis of a specific protein before and after stimulation can be used to discern whether new protein sites are phosphorylated after a cell has been stimulated.

### EXAMPLE 44

Identification and Assays for Tyrosine Protein Kinases

The present invention includes the discovery of tyrosine protein kinases (TPKs). These enzymes contribute to cellular signal transduction. Using immune-complex protein kinase assays, the presence or absence of a given TPK can be established and an estimation of its functional state obtained. In this method, TPKs are immunoprecipitated, allowed to autophosphorylate in the presence of labeled ATP, run out on an SDS-PAGE gel, and detected by autoradiography. The alternate methods assess the functional state of TPKs by providing a potential substrate along with the labeled ATP in the reaction mixture. For example, the exogenous substrate is a protein (enolase or a-casein) permitting simultaneous assessment of autophosphorylation and exogenous substrate phosphorylation, or a peptide substrate, resulting in a rapid, high-throughput assay that evaluates only exogenous substrate phosphorylation. This method provides an assay for TPK function that allows simultaneous analysis of a large number of samples. It utilizes the synthetic peptide RR-SRC (Arg-Arg-Leu-Ile-Glu-Asp-Ala-Glu-Tyr-Ala-Ala-Arg-Gly), which represents the phosphorylation site in pp60^{src} with two arginine residues added to the amino terminus and can be used as a substrate for the immunoprecipitated TPK.

These procedures can be used with lymphoid or nonlymphoid cells, primary cells or cultured cell lines, and activated or nonactivated cells. Cells of interest are collected, activated if desired, and the cellular proteins solubilized in a detergent-containing buffer. Tyrosine protein kinases are immunoprecipitated with an anti-TPK antibody and the resulting immune complexes washed to remove other proteins. [g³²P]ATP and an appropriate divalent cation are added, leading to radioactive labeling of the kinase through autophosphorylation. The reaction mixture is fractionated using SDS-PAGE and the labeled products detected by autoradiography.

### EXAMPLE 45

Assessment of the Effects of Tyrosine Protein Kinase Inhibitors

The present invention includes the discovery of inhibitors for tyrosine protein kinases. Inhibition of TPKs provides an important means of controlling many signaling pathways in mammalian cells in normal and pathological conditions. An assay may be used to test the inhibitory effects of TPK inhibitors in vitro on a specific TPK that has been immune-precipitated from cell lysates (as described in UNIT 11.4. in the Current Protocol in Immunology, John E. Coligan et al.). Another assay may be used to test the effects of several TPK inhibitors on TPKs in vivo in activated cells. Parameters such as the quantities of cells and antiserum and the choice of divalent cation can be optimized depending on the cell line used in the experiment and the TPK it produces. These methods can be used to assay the effects of inhibitors on receptor TPKs as well.

### EXAMPLE 46

Assessing Tyrosine Protein Kinase Inhibitors Using Intact Cultured Mammalian Cells

In this type of assay mammalian cells are treated with TPK inhibitors followed by a cell stimulation strategic (often involving the addition of an activating antibody or growth-inducing cytokine). Different endpoints may then be used to measure the effect of an inhibitor. The method utilizes a relatively broad indicator of TPK-dependent signal transduction: the pattern of cellular protein reactivity with antiphosphotyrosine as detected by immunoblotting (UNIT 11.3, in the Current Protocol in Immunology, John E. Coligan, et al.). This procedure could be applied to any type of mammalian cells.

### EXAMPLE 47

Macrophage Activation and Functions

Specific antibodies to particular receptors on macrophage cell lines, for example, can activate these cells and increase their functional capacity toward pathogens, and/or infected cells with pathogens, and/or tumor cells. Therefore, the present invention includes the measurement of the effect of antibodies on the ability of macrophages to phagocytose particles (Metchnikoff, 1884, 1893) and the elimination of intruding microorganisms, tumor cells, and cellular debris, which is mediated through distinct surface receptors, among them lectin-like receptors that recognize carbohydrates on microbial surfaces (Gordon et al. 1988), vitronectin (Savill et al. 1990) and other specialized receptors that recognize apoptotic cell surfaces, and various complement receptors (Drevets and Campbell, 1991), as well as other specialized molecules. For example, one of the complement receptors, CR3, binds directly to complement components C3bi and C3d on the surface of various microorganisms (Drevets and Campbell, 1991), possibly through direct binding to bacterial LPS. Phagocytosis can be dramatically enhanced through the use of receptors for the Fc region of immunoglobulins bound to targets such as tumor cells (Kinet 1989).

Macrophages are activated with low levels of the cytokine IFN-g for 4 hr followed by triggering either with higher levels of IFN-g or with LPS for a short time period. Production of reactive nitrogen intermediates (RNI; UNIT 14.5, as described in the Current Protocol in Immunology (CPI), John E. Coligan., et al.) or cytotoxicity against intracellular parasites or bacteria or other pathogens can then be measured after an appropriate culture period (48 to 72 hr) as described in UNIT 14.6. (CPI). Many intra- and extracellular targets can act as trigger signals for primed macrophages, in such instances LPS can be replaced with the target as trigger signal.

### EXAMPLE 48

Activation or Inhibition of Inflammatory Macrophages

The present invention involves the measurement of macrophage-mediated cytotoxicity against tumor cells, using inflammatory macrophages cultured as adherent cells (see UNIT 14.7/CPI)., and specific antibodies raised against gene products. This method refers to a procedure for determining the ability of macrophages from a mammal to lyse tumor cells in vitro. The assay includes: (1) culture of macrophages (freshly explanted from vertebrate as described in UNIT 14.1/CPI or grown from a cell line) with suspected activating reagents for 18 hr in a 96-well microtiter plate; (2) extensive washing of the cultured macrophages to remove residual reagents, followed by 24-hr incubation with [¹¹¹In]-labeled tumor cells to allow lysis to occur; and (3) collection of cell-free culture supernatants and measurement of cytolytic activity as a function of ¹¹¹In released from tumor cells destroyed by activated macrophages.

### EXAMPLE 49

Antibody Effects on Oxidative Metabolism of Macrophages from Vertebrate

The present invention includes the assessment of macrophage cytotoxicity as judged by enhanced production of reactive nitrogen and oxygen intermediates such as inorganic nitrite (NO₂⁻) and superoxide anion (O₂⁻). This microassay can be used to measure the levels of NO₂⁻ and O₂⁻ respectively that are generated by antibodies of immunologically-stimulated macrophages. Detection of these products may be used to identify cytokine(s), microbe(s), or microbial products(s) that regulate oxidative metabolism and effector activity.

### EXAMPLE 50

Colorometric Determination of Macrophage Killing of Bacteria

Bacteria and cells are mixed in a microtiter plate and extracellular organisms are removed by washing. The numbers of surviving bacteria are determined before and after a 90-min incubation by measuring their ability to reduce the yellow tetrazolium salt, 3-(4,5-dimethylthiazol-2-yl)2,5-diphenyltetrazolium bromide (MTT) to a purple formazan product. This reaction, which is mediated by bacterial dehydrogenases, can be detected colorimetrically by reading the plates on a microtiter plate reader at 570 nm and comparing the results to a standard curve. This assay is a modification of the one originally described by Peck (1985).

### EXAMPLE 51

Effect of Antibodies on Antiviral Activity of Mammalian Cells

The present invention includes the assessment of the effect of antibody on the production and secretion of substances that possess antiviral effect, such as interferons, by mammalian cells like macrophages, and resistance to the cytopathic effect (CPE) of viral infection.

This method provides a qualitative assay of the presence or absence of endogenous antiviral substance production in mammalian macrophages by measuring the resistance of macrophages to the CPE of infection with a particular virus. For example, macrophages are placed in 96-well tissue culture plates and allowed to adhere. They are then challenged with a viral suspension. After approximately 24 hours, the virus is removed and the monolayer is stained with crystal violet, which detects cell death (CPE) as a decrease in staining intensity.

### EXAMPLE 52

Enhancement or Inhibition of Enzymatic Activity: Effect of Antibodies on Enzymatic Activity

The specific binding between antibodies and a gene product in the cell of a mammal can result in changes at different magnitude in the mammalian cell, including changes in particular enzymatic activity. For example, enzyme induction occurs in response to disease and can be useful in either diagnostic applications for drug discovery. The present invention includes the assessment of enzyme production or inhibition in pathological and normal conditions, using colorimetric tests. For example, the measurement of alkaline phosphatase, a liver enzyme, as well as other liver enzymes will indicate the disease status of this organ.

Another important enzyme, the creatine kinase isoform, is a critical marker for myocardial and skeletal muscle diseases, whose modulation can lead to therapeutic alternatives to these diseases. Angiotensin-converting enzyme that catalyses the conversion of angiotensin I to angiotensin beta, is a vital enzyme in the control of aldosterone secretion and blood pressure. The control of this enzyme is an effective therapy against the blood pressure. Therefore, the present invention includes the use of antibodies and multiple mammalian cell lines to control or shift the production of critical enzymes in the cell.

### EXAMPLE 53

Enhancement or Inhibition of Enzymatic Activity: Effect of Antibodies on Enzymatic Activity

The specific binding between antibodies and a gene product in the cell of a mammal can result in changes at different magnitude in the mammalian cell, including changes in particular enzymatic activity. For example, enzyme induction occurs in response to diseases and can be useful in either diagnostic applications or for drug discovery. The present invention includes the assessment of enzyme production or inhibition in pathological and normal conditions, using calorimetric tests. For example, the measurement of alkaline phosphatase, a liver enzyme, as well as other liver enzymes will indicate the disease status of this organ. Another important enzyme, the creatine kinase isoform, is a critical marker for myocardial and skeletal muscle diseases, whose modulation can lead to therapeutic alternatives to these diseases. Angiotensin-converting converting enzyme that catalyses the conversion of angiotensin I to angiotensin II, is a vital enzyme in the control of aldosterone secretion and blood pressure. The control of this enzyme is an effective therapy against the blood pressure. Therefore, the present invention includes the use of antibodies and multiple mammalian cell lines to control and/or shift the production of critical enzymes in the cell.

### EXAMPLE 54

Anti-idiotype Activity in Infectious Diseases

The present invention includes the use of antibodies to particular antigens specific for pathogens, to cancer and other diseases in an anti-idiotypic strategy for therapy. For example, anti-idiotypic antibodies to tumor specific marker can induce specific immune response against the tumor cells. Alternatively, the anti-idiotypic antibody may down-regulate the growth of the tumor via the idiotypic network. Additionally, the anti-idiotypic antibody can also be used as a vaccine. Therefore, specific antibodies produced according to the present invention will be used to identify anti-idiotypic antibodies with specific physiological reaction to particular pathologic conditions.

### EXAMPLE 55

Mimotope Receptor Using Phage Display

The present invention includes the use of recombinant phage technology, as described previously U.S. Pat. Nos. 4,797,363 Teodorescu et al., 4,987,073 Berman et al., 5,223,409 Ladner et al., and 5,338,665 Schatz et al., to design novel drugs to control pathological conditions. For example, anti-gene product antibodies that bind to cell receptors of tumor cells and lead to cell death will be used to find specific mimotopes using a recombinant phage with unknown sequences to inhibit the interaction of the receptor and the effective antibody. The mimotope can be analyzed at the molecular level and used to design effective drugs against the tumor cell. This approach is not limited to mammalian tumor cells. The assay uses the antibodies (GPTs) to select the competitive recombinant phage toward the receptor, then uses the recombinant phage to define the ligand, and finally uses the defined ligand to design a drug that can act specifically on the said receptor.

### EXAMPLE 56

Use of Antibodies as a Drug Antagonists

The present invention includes the use of antibodies to select a drug antagonist and discover their action on a particular receptor.

### EXAMPLE 57

Zymogram Assays

The present invention includes assays to identify newly synthesized enzymes in normal and pathological conditions. For example, tumor cells synthesize particular enzymes that are either typical for the tumor cells or are increased in their synthesis as compared to their counterpart in normal cell lines. In these cases, a zymogram can be used to assess the effect of antibodies on mammalian cell. Culture medium and mammalian cell extracts from mammalian cells that have been incubated with antibodies against gene products are analyzed on acrylamide/bis acrylamide, glycine, Tris-base gels. Extracts are run on a zymogram gel using Tris-base, Glycine SDS running buffer. When the run is complete, the gel is soaked in renaturing buffer which contains a non-ionic detergent. The gels are then equilibrated in a developing buffer which returns a divalent metal cation required for enzymatic activity. The gel is then stained in coomassie to visualize clear bands where the enzymes have digested the gelatin.

All of the patents, publications, and references disclosed herein are specifically incorporated by reference as though fully set forth herein.

There will be various modifications, improvements, and applications of the disclosed invention that will be apparent to those of skill in the art, and the present application encompasses such embodiments to the extent allowed by law. Although the present invention has been described in the context of certain preferred embodiments, the full scope of the invention is not so limited, but is in accord with the scope of the following claims.

What is claimed is:

1. A method to identify the function of a gene product in a selected biological process or physiological condition comprising:
selecting at least one polynucleotide derived from human DNA, wherein the gene product of the polynucleotide does not have a known function in the selected biological process or physiological condition,
delivering the gene product of the polynucleotide to the immune system of a non-human mammal to produce an immune response to the gene product,
obtaining sera from the non-human mammal, wherein the sera contains antibodies specific to the gene product of the polynucleotide, and
testing the antibodies in an immunoassay to determine the function of the gene product in the selected biological process or physiological condition.

2. The method of claim 1 wherein the gene product of the polynucleotide is delivered to the immune system of the mammal and expressed in vivo by incorporating the polynucleotide into a plasmid construct that is administered to the mammal.

3. The method of claim 1 wherein the gene product is expressed in a prokaryotic expression system.

4. The method of claim 3 wherein the gene product is expressed in vitro followed by administration of the gene product to the mammal.

5. The method of claim 4 further comprising the step of purifying the gene product prior to administration to the mammal.

6. The method of claim 3 wherein the prokaryotic expression system is administered to the mammal.

7. The method of claim 1 wherein the immunoassay is selected from the group consisting of ELISA, immunofluorescence, immunostaining, flow cytometry, antibody-dependent cell-mediated cytotoxicity, chemotaxis assay, Elispot assay, and anti-idiotypic assay.

8. The method of claim 1 wherein the immunoassay measures tumor cell growth.

9. The method of claim 1 wherein the immunoassay identifies a cell surface marker on a tumor cell.

10. The method of claims 1, 2, 3, 4, 5, 6, 7, 8, or 9 further comprising sequencing at least one of the polynucleotide and the gene product.

11. A method to identify the function of a gene product in a specific pathological condition comprising:
selecting at least one first polynucleotide derived from human DNA from a normal tissue or cell,
delivering the gene product of each of the first and second polynucleotide to the immune system of a mammal to produce an immune response to the gene product,
obtaining sera from the mammal, wherein the sera contains antibodies specific to the gene product of the first and second polynucleotide, and
testing the antibodies in an immunoassay to determine the function of the gene product of the first or second polynucleotide in the selected biological process or physiological condition.

12. The method of claim 11 wherein the gene product of either of the first or second polynucleotide is delivered to the immune system of the mammal by incorporating the polynucleotide into a plasmid construct that is administered to the mammal and expressed in vivo.

13. The method of claim 11 wherein the gene product of either the first or second polynucleotide is expressed in a prokaryotic expression system.

14. The method of claim 13 wherein the expression of the gene product of either of the first or second polynucleotide occurs in vitro followed by administration of the gene product to the mammal.

15. The method of claim 14 further comprising the step of purifying the gene product prior to administration to the mammal.

16. The method of claim 13 wherein the prokaryotic expression system is administered to the mammal.

17. The method of claim 11 wherein the immunoassay is selected from the group consisting of ELISA, immunofluorescence, immunostaining, flow cytometry, antibody-dependent cell-mediated cytotoxicity, chemotaxis assay, Elispot assay, and anti-idiotypic assay.

18. The method of claim 11 wherein the immunoassay measures tumor cell growth in the presence of antibodies specific to gene product of the second polynucleotide.

19. The method of claim 11 wherein the immunoassay identifies a cell surface marker on a tumor cell.

20. The method of claims 11, 12, 13, 14, 15, 16, 17, 18, or 19 further comprising sequencing at least one of the first or second polynucleotide and the gene product of the first or second polynucleotide.
